(19) United States
(12) Patent Application Publication
(10) Pub. No.: US 2009/0222068 A1
(43) Pub. Date: Sep. 3, 2009

(54) RAPID FLASH OPTICAL THERAPY

(75) Inventors: Richard Oberreiter, Newport Beach, CA (US); James Harry Kraushaar, Newport Beach, CA (US); Bruce Sargeant, Orange, CA (US); Aaron Nissen, La Habra, CA (US); Tim Duffy, Anaheim Hills, CA (US)

Correspondence Address: KNOBBE MARTENS OLSON & BEAR LLP, 2040 MAIN STREET, FOURTEENTH FLOOR, IRVINE, CA, 92614, US

(73) Assignee: CLRS TECHNOLOGY CORPORATION, Costa Mesa, CA (US)

(21) Appl. No.: 12/393,995

(22) Filed: Feb. 26, 2009

Related U.S. Application Data
(60) Provisional application No. 61/032,933, filed on Feb. 29, 2008.
(60) Provisional application No. 61/121,492, filed on Dec. 10, 2008.

(51) Int. Cl. A61N 5/06

(52) U.S. Cl. 607/88

(57) ABSTRACT

Systems and methods are disclosed for maintaining a therapeutically significant level of energy at a treatment area that can include rapid charging of a storage element and triggering of a flashlamp by discharging the storage element and repeating the charging and triggering a predetermined number of times during a treatment period to raise and maintain a temperature at the treatment area to a predetermined therapeutic level.

### CROSS-REFERENCE TO RELATED APPLICATIONS

The present application claims priority benefit under 35 U.S.C. §119(e) from U.S. Provisional Application No. 61/032,933, filed Feb. 29, 2008, and from U.S. Provisional Application No. 61/121,492, filed Dec. 10, 2008, which are incorporated by reference herein.

### BACKGROUND

1. Field

The disclosure generally relates to devices, methods, and systems, for treating skin conditions. For example, with respect to some embodiments, the disclosure relates to a light emitting, therapeutic, rapid-flash device for the treatment of skin conditions, including acne.

2. Description of the Related Art

Skin conditions can cause serious health risks, and in extreme cases can lead to scarring, embarrassment, or even psychological damage. One of the most common skin conditions is acne, the most common form being acne vulgaris. Acne affects millions of people in the United States and is an inflammatory disease caused generally as a result of blockages in hair follicles. Acne affects the face and upper neck most commonly, but other areas of the body may also develop acne blemishes. While acne most commonly affects people during adolescence, it can affect people of all ages.

There is significant demand for skin treatment devices, particularly for those that treat acne. Several acne treatment methods are known including topical bactericidal products, topical antibiotics, oral antibiotics, hormonal treatments, topical retinoids and oral retinoids. Less common treatment methods include the use azelaic acid, zinc, tea tree oil, nicotinamide, and other agents. However, these products often have undesirable side effects, or have limited results.

Devices have also been used to treat acne, but the equipment is often large, expensive and difficult to use. There is therefore a need for a safe, user-friendly, hand-held, light emitting therapeutic device to treat skin conditions including acne.

### SUMMARY

In one embodiment, a method for maintaining a therapeutically significant level of energy at a treatment area includes determining current provided to a primary winding of a fly-back transformer, the fly-back transformer comprising said primary winding and a secondary winding. The method further involves charging an energy storage device by discharging energy from said secondary winding into said energy storage device when said measured current is within a predetermined range. The method further comprises repeating said determining and said discharging until stored energy with said energy storage device is within a predetermined discharge range. The method also includes triggering a flashlamp when said stored energy is within the predetermined discharge range such that said stored energy is discharged across said flashlamp. The method involves emitting light from said flashlamp into a treatment area as a result of said triggering, said light comprising a plurality of wavelengths in a range of from about 400 nm to about 1100 nm. The method comprises repeating said charging and triggering a predetermined number of times during a treatment period to raise and maintain a temperature at said treatment area to a predetermined therapeutic level.

In some embodiments, the energy storage device comprises at least one capacitor. The fly-back transformer may further comprise a core, and wherein said predetermined range corresponds to flux saturation of said core. The predetermined discharge range comprises about 300 Vdc, for example. In some embodiments, the triggering comprises ionizing gas contained in said flashlamp. The light may comprise a plurality of wavelengths in a range of from about 400 nm to about 700 nm. The predetermined number of times is six times in some embodiments. The treatment period may comprise about two seconds in some embodiments. The treatment area can include tissue containing Acne Vulgaris bacteria. The predetermined therapeutic level is about 49 degrees Celsius in some embodiment. In some embodiments, the predetermined therapeutic level is at least 49 degrees Celsius.

In some embodiments the repeating said charging and triggering is sufficient to raise and maintain an energy density at the treatment area to a predetermined therapeutic energy density. In some embodiments, the predetermined therapeutic energy density is about 6 J/cm2 at the treatment area. The predetermined therapeutic energy density may comprise at least 6 J/cm2 at the treatment area.

In one embodiment, a device configured to maintain a therapeutically significant level of energy at a treatment area includes a fly-back transformer, comprising a primary winding, a secondary winding, and a core. The device also includes an energy storage device configured to receive energy from said secondary winding when detected current into said primary winding reaches a predetermined level.

The device further comprises a controller that is configured to determine when energy stored with said energy storage device reaches a predetermined discharge level, said controller further configured to cause additional current to be directed to said primary winding until said stored energy reaches said predetermined discharge level.

The device includes a flashlamp, wherein said controller is further configured to discharge said stored energy across said flashlamp when said stored energy reaches said predetermined discharge level, and wherein said flashlamp is configured to emit light comprising a plurality of wavelengths in an optical range of from about 400 nm to about 1100 nm, wherein said controller is further configured repeat delivery of energy to said energy storage device and to repeat discharge of said energy across said flashlamp a predetermined number of times during a treatment period to raise and maintain a temperature at a treatment area to a predetermined therapeutic level.

The device further includes a user input in some embodiments wherein said predetermined therapeutic level is determined based on said user input. The controller may be configured to determine said predetermined therapeutic level based on said user input. The controller comprises a microprocessor in one embodiment. The controller comprises discreet logic in some embodiments.

The light may include a plurality of wavelengths of from about 400 to about 700 nm. The predetermined number of times is six times in certain embodiments. The treatment period may include about two seconds. The device of claim 15, wherein said treatment area comprises Acne Vulgaris bacteria. The predetermined therapeutic level is about 49 degrees Celsius in an embodiment. In an embodiment, the predetermined therapeutic level is at least 49 degrees Celsius.

The controller may be further configured to raise and maintain an energy density at the treatment area to a predetermined therapeutic energy density. In some embodiments, predetermined therapeutic energy density is about 6 J/cm2 at the treatment area. The predetermined therapeutic energy density may be at least 6 J/cm2 at the treatment area.

In another embodiment, a light-emitting therapeutic device for treating acne includes a fly-back transformer, an energy storage device coupled to an output of said fly-back transformer, and a flashlamp coupled to said energy storage device. The device further includes a controller configured to flash said flashlamp, by delivering energy from said energy storage device to said flashlamp, a sufficient number of times within a predetermined treatment period to raise the temperature of a treatment area having acne to at least 49 degrees Celsius during said treatment period. The sufficient number of times may be six times, for example. The predetermined treatment period can be about two seconds.

In one embodiment, a method of treating acne with a light-emitting therapeutic device involves charging an energy storage device using a fly-back transformer, triggering a flashlamp to discharge energy stored with said energy storage device across said flashlamp, wherein said triggering causes said flashlamp to emit therapeutic light, and repeating said charging and triggering a sufficient number of times within a predetermined treatment period to raise the temperature of a treatment area having acne to at least 49 degrees Celsius during said treatment period with said therapeutic light. The sufficient number of times may be six times, for example. In an embodiment, the predetermined treatment period is about two seconds.

In another embodiment, a light emitting therapeutic device for maintaining a therapeutically significant level of energy at a treatment area on a user's body includes a broadband light source, the light source configured to generate optical energy having a wavelength in a range of from about 400 nm to about 1100 nm. The device further includes a rapid flash unit, the rapid flash unit configured to activate the broadband light source a predetermined number of times to raise and maintain a treatment site's temperature at a therapeutic treatment level.

The rapid flash unit comprises an energy storage device, configured to provide energy to the light source, a transformer, electrically coupled to the storage device, wherein the transformer is configured to provide energy to the energy storage device when activated and a controller.

The controller is in electrical communication with the transformer and the storage element, the controller configured to monitor current received by the transformer and activate the transformer to send energy to the storage element when sufficient current is detected, the controller further configured to monitor the charge stored with the storage element and to re-activate the transformer until a desired charge is stored with the storage element, the controller further configured to activate the broadband light source by discharging the desired charge across the light source, wherein said controller is further configured repeat the sending of the energy to the storage element and the activation of the light source a predetermined number of times during a treatment period such that the treatment site's temperature is raised to the therapeutic treatment level. The transformer may be a flyback transformer, for example. In an embodiment, the storage element comprises at least one capacitor. The light source may include a flash lamp, for example. The light source may further comprise an LED. In some embodiments, the wavelength is in a range of from about 400 nm to about 700 nm. The treatment site comprises acne vulgaris bacteria in some embodiments. The predetermined treatment temperature is about 49 degrees Celsius, for example. In an embodiment, the predetermined treatment temperature is at least 49 degrees Celsius.

The transformer may comprise a primary coil, a secondary coil, and a core, wherein the controller is configured to charge the storage element by delivering a plurality of pulses of current to the primary coil, each of the plurality of pulses of current causing the secondary coil to deliver a partial charge to the storage element. The controller may be configured such that each of the plurality of pulses of current at least nearly saturates the core. The controller may be configured such that each partial charge is of substantially equal magnitude and wherein the controller is configured such that the partial charges are delivered to the storage element at a substantially constant frequency.

The device can further include a user interface, configured to be placed into contact with the treatment area and to transmit the optical energy from the light source to the treatment area, wherein the controller is configured to allow light source activation based at least in part on an orientation condition of the user interface with respect to the treatment area. The user interface of certain embodiments, comprises an impedance sensor configured determine when the user interface is contacting the treatment area, and wherein the controller is configured to receive at least one sensor signal from the electrical impedance sensor and determine the orientation condition based on the at least one sensor signal. The user interface can define a transmission pathway of the optical energy from the light source to the treatment area, wherein the user interface comprises a first contact sensor and a second contact sensor spaced apart from the first contact sensor, and wherein a linear path from the first contact sensor to the second contact sensor at least partially traverses the transmission pathway, and wherein the controller is configured to determine the orientation condition based on whether both the first and second contact sensors are in contact with the treatment area. In some embodiments, the user interface comprises an output window, and at least two contact sensors, wherein the controller is configured determine the orientation condition based on an angular alignment between the output window and the treatment area prior to delivering optical energy to the treatment area, wherein controller is further configured to determine the angular alignment based on at least two sensor signals received from the at least two the contact sensors.

In one embodiment, a method of maintaining for maintaining a therapeutically significant level of energy at a treatment area on a user's body involves providing a light emitting device. The light emitting device includes a broadband light source, the light source configured to generate optical energy having a wavelength in a range of from about 400 nm to about 1100 nm. The light emitting device also includes a rapid flash unit, the rapid flash unit configured to activate the broadband light source a predetermined number of times to raise and maintain a treatment site's temperature at a therapeutic treatment level.

The rapid flash unit includes an energy storage device, configured to provide energy to the light source, a transformer, electrically coupled to the storage device, wherein the transformer is configured to provide energy to the energy storage device when activated, and a controller, in electrical communication with the transformer and the storage element.

The method further includes charging the storage element, wherein the charging involves monitoring the current received by the transformer. The method further includes activating the transformer to send energy to the storage element when sufficient current is detected, monitoring the charge stored with the storage element, and re-activating the transformer when a desired charge is stored with the storage element. The method further includes activating the broadband light source by discharging the desired charge across the light source. The method also involves repeating the charging and activating a predetermined number of times during a treatment period such that the treatment site's temperature is raised to the therapeutic treatment level. The method may further include detecting when the charge is sufficient to activate the light source before activating the light source. The transformer comprises a flyback transformer in some embodiments. The storage element comprises a capacitor in some embodiments, for example.

The generating of the charge can include delivering a plurality of pulses of current to a primary coil of the transformer and delivering a plurality of partial charges to the capacitor, each of the plurality of partial charges generated in response to one of the plurality of pulses of current. The delivering the plurality of pulses of current can include, for each of the plurality of pulses of current, delivering enough current to the transformer to at least nearly saturate the core of the transformer. Delivering the plurality of partial charges comprises delivering partial charges of substantially equal magnitude and delivering the partial charges at a substantially constant frequency in certain embodiments.

The method can further include filtering the optical energy prior to delivery to the treatment area. The filtering removes energy having a wavelength outside of a range of from about 400 nm to about 700 nm, in some embodiments.

The light emitting device of certain embodiments further comprises a user interface, configured to be placed into contact with the treatment area and configured to transmit the optical energy from the light source to the treatment area. The method can further include determining an orientation condition of the user interface with respect to the treatment area and allowing light source activation based at least in part on an orientation condition of the user interface with respect to the treatment area.

The user interface can be configured to provide a transmission pathway of the optical energy from the light source to a treatment area, wherein the user interface comprises an impedance sensor.

The method can further include generating an impedance signal with the electrical impedance sensor and determining the orientation condition based on when the impedance signal indicates that the user interface is in contact with the treatment site.

The user interface of certain embodiments is configured to provide a transmission pathway of the optical energy from the light source to a treatment area, wherein the user interface comprises a first contact sensor and a second contact sensor spaced apart from the first contact sensor, wherein a linear path from the first contact sensor to the second contact sensor at least partially traverses the transmission pathway.

The method can further include determining a contact signal with the contact sensors and determining the orientation condition based on the contact signal.

The user interface of certain embodiments is configured to transmit the optical energy from the light source to a treatment area, the user interface comprising an output window, and at least two sensors.

The method can further include receiving at least two sensor signals from the at least two sensors, determining an angular alignment between the output window and the treatment area based upon the at least two sensor signals and determining the orientation condition based on the angular alignment.

In one embodiment, a light emitting device for providing therapy to a user includes a light source, a heat source, and a user interface. The device may additional include a controller to control the heat source. The same controller, or a different controller, may be provided to control the light source. The device is configured to provide a therapeutic dosage of heat and light energy to a treatment site on a patient. For example, the device can be configured to provide enough heat and light energy to treat acne. In all embodiments, the heat source can be a separate component from the light source. For example, in some cases, the light source is light bulb or light-emitting diode, and the heat source is a resistive element, such as a wire or electrically conductive film having a resistance suitable for heating.

The device can include a cooling device to cool the user interface. The cooling device can be active or passive, such as a fan, thermoelectric cooler, cooling fins, or a combination of one or more active and passive cooling devices.

In one embodiment, a phototherapeutic method of treating acne comprises delivering therapeutic dosages of heat and light to a treatment site. The heat may be delivered to a window through which light from a heat source is emitted. For example, in one embodiment, a heat source is activated to provide heat to an output window, the heated window is placed in contact with a treatment area (e.g., an area on a user's skin that is affected by acne), and light is transmitted through the window to the treatment area.

In one embodiment, a light emitting device for providing therapy to a user is provided. The device includes a light source, configured to generate optical energy having a wavelength in a range of from about 400 nm to about 1200 nm; a user interface, configured to be placed in contact with a treatment area on a user's body and configured to transmit optical energy from the light source to the treatment area; and a heat source coupled to the user interface.

The heat source may be configured to heat the user interface prior to, during, and/or after activation of the light source. The heat source may include a resistive heating element. The user interface may include a transmission window and the resistive heating element may be attached to a light-transmitting surface of the transmission window or to a perimeter surface of the transmission window. In some embodiments, the resistive heating element is attached to the transmission window such that the resistive heating element at least partially blocks light traveling through the transmission window.

The light source can include a flashlamp, an LED, or a combination. The light source can be configured to emit light having a wavelength in a range of from about 400 nm to about 700 nm. The user interface can include a transmission window configured to filter the optical energy prior to transmission to the treatment area.

The light emitting device may also include a second light source configured to generate second optical energy having a second wavelength. The light emitting device may also include an aiming beam, configured to illuminate the treatment area prior to activation of the light source.

The light emitting device may also include a button that defines a button depression axis, wherein the button depression axis is substantially aligned with a beam propagation axis, and wherein the light source is configured to transmit optical energy generally along the beam propagation axis.

The user interface may define a transmission pathway through an opening in the user interface, wherein the user interface also includes a locating ridge positioned at least partially around the opening and is configured to provide tactile feedback to a user regarding the position of the opening. The locating ridge can extends around the entire opening. The user interface may also include at least one contact sensor configured to determine when said user interface is in contact with the treatment area.

In another embodiment, a light emitting device for providing therapy to a user includes a light source, configured to generate optical energy having a wavelength in a range of from about 400 nm to about 1200 nm; an output window, configured to be placed in contact with a treatment area on a user's body and configured to at least partially transmit the optical energy from the light source to the treatment area; and a heating element attached to a surface of the output window and configured to deliver heat energy to the output window, wherein the light source and output window are aligned such that light emitted from the light source at least partially travels through the heating element prior to delivery to the treatment area.

The heating element may include a resistive heating element. The output window may include an input surface and an output surface configured to contact the treatment area, wherein the heating element is attached to the input surface. The heating element may extend in a zig-zag pattern and define gaps between portions of the heating element.

The light source may include a flashlamp or an LED. The wavelength may be in a range of from about 400 nm to about 700 nm. The output window may include a filter configured to filter the optical energy prior to transmission to the treatment area. The light emitting device may also include a second light source configured to generate second optical energy having a second wavelength. The second light source may include an aiming beam, configured to illuminate the treatment area prior to activation of the light source.

The light emitting device may also include a button configured to activate the light emitting device, wherein the button defines a button depression axis, wherein the button depression axis is substantially aligned with a beam propagation axis along which optical energy from the light source is aligned to travel.

The user interface may define a transmission pathway through an opening in the user interface, and the user interface may also include a locating ridge positioned at least partially around the opening and may be configured to provide tactile feedback to a user regarding the position of the opening. The user interface may also include a contact sensor configured to determine when the user interface is in contact with the treatment area.

In yet another embodiment, a phototherapeutic method of treating acne includes: heating an output window with a heat source; and emitting light from a light source through the output window towards a treatment area on a user's body, wherein the emitting includes generating light having a wavelength in a range of from about 400 nm to about 1200 nm.

The heating may be performed prior to, during, and/or or after said emitting. The heating may include heating the output window with a resistive heating element attached to a surface of the output window. The method may also include partially obstructing the light with the resistive heating element.

The emitting may include emitting light with a flashlamp and/or an LED. The emitting may include emitting light with a wavelength in a range of from about 400 nm to about 700 nm.

The method may also include filtering said light. The method may also include sensing when the output window is in contact with the treatment area. The method may also include enabling activation of the light source based upon the sensing.

For purposes of summarizing the invention, certain aspects, advantages and novel features have been described herein. Of course, not necessarily all such aspects, advantages or features will be embodied in any particular embodiment.

### BRIEF DESCRIPTION OF THE DRAWINGS

These and other features will now be described with reference to the drawings summarized below. These drawings and the associated description are provided to illustrate certain embodiments, and not to limit the scope of the invention.

FIG. 1 is a block diagram of an acne treatment device according to an embodiment of the disclosure;

FIGS. 2A-C are front perspective, rear perspective and right side views, respectively, of an acne treatment device according to an embodiment of the disclosure, such as the embodiment of FIG. 1 or the embodiment of FIG. 18;

FIG. 3 is a block diagram of a rapid flash unit of an acne treatment device according to one embodiment of the disclosure, such as the embodiments of FIGS. 1, 2 or 18;

FIG. 4 is a functional schematic diagram of a rapid flash unit of an acne treatment device according to another embodiment of the disclosure, such as the embodiments of any of FIGS. 1-3 or 18;

FIG. 5 is a schematic diagram of a rapid flash unit of an acne treatment device according to one embodiment of the disclosure, such as the embodiments of any of FIGS. 1-4 or 18;

FIG. 6 shows an embodiment of a method of treating acne with an acne treatment device having a rapid flash unit according to one embodiment of the disclosure, such as with any of the devices of FIGS. 1-5 or 18;

FIG. 7 shows an embodiment of a method of rapidly charging the acne treatment device of FIG. 6;

FIG. 8 shows a timing chart of a rapid flash unit of an acne treatment device according to another embodiment of the disclosure, such as any of the embodiments of FIGS. 1-7 or 18;

FIGS. 9A-B are right side and top views, respectively, of an acne treatment device according to another embodiment of the disclosure, such as any of the embodiments of FIGS. 1-5 and 18;

FIGS. 10A-E are front perspective, right side, and three front perspective views, respectively, of several acne treatment devices according to various embodiments of the disclosure, such as any of the embodiments of FIGS. 1-5, 9A-B and 18;

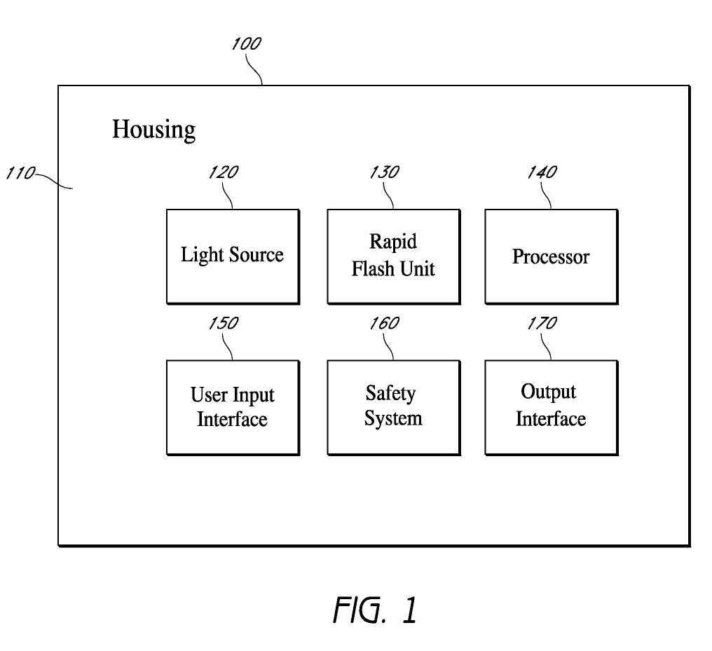

FIGS. 1A-B are cross-sectional views of output interfaces of an acne treatment device according to embodiments of the disclosure, such as any of the embodiments of FIGS. 1-5, 9A-10E and 18;

FIGS. 12A-E are front views of the head portion of an acne treatment device including safety mechanisms including sensor arrays according to various embodiments of the disclosure, such as any of the embodiments of FIGS. 1-5, 9A-11B and 18; and

FIG. 13 shows a schematic diagram of a capacitive contact sensor circuit according to one embodiment of the disclosure;

FIGS. 14-17 show various embodiments of methods of treating acne with an acne treatment device according to various embodiments of the disclosure that can be implemented using any of the devices of FIGS. 1-5, 9A-12 and 18.

FIG. 18 is a block diagram of an acne treatment device according to an embodiment of the disclosure;

FIG. 19 is a block diagram of an output interface compatible with any of the acne treatment devices and methods described herein, including those of FIGS. 1, 2, 9A-B, 10A-E, 11A-B, 12A-E, 13-16, 18 and 25-28;

FIG. 20 is a partial cross-sectional side view of an output interface having a heat source indirectly coupled to an output window, according to one embodiment;

FIG. 21 is a partial top view of an output interface having multiple heat sources indirectly coupled to an output window, according to another embodiment;

FIG. 22 is a partial side view of an output interface having a heat source directly coupled to an output window, according to another embodiment;

FIGS. 23A-C are partial top views of output interfaces showing different heating element configurations, according to various embodiments;

FIGS. 24A-B illustrate embodiments of methods of treating acne with an acne treatment device having a light source and a heat source, according to various embodiments;

FIGS. 25-28 show various embodiments of methods of treating acne with an acne treatment device according to various embodiments of the disclosure.

### DETAILED DESCRIPTION

A light emitting therapeutic device 100 in accordance with one embodiment of the present disclosure is illustrated in FIG. 1. In one embodiment, the device 100 is a hand-held, ergonomically designed unit that allows a user to treat him or herself The device 100 may also be described as a self-contained, hand-held, portable unit that is configured to be carried by a user. For example, in various embodiments, the device 100 is configured to be carried in the user's pant, shirt, or jacket pocket, or within a purse, handbag, or backpack. Embodiments described herein generally relate to embodiments of light emitting therapeutic devices described in U.S. patent application Ser. No. 11/951,240 (“the '240 Application), filed Dec. 5, 2007 which is incorporated in its entirety by reference herein.

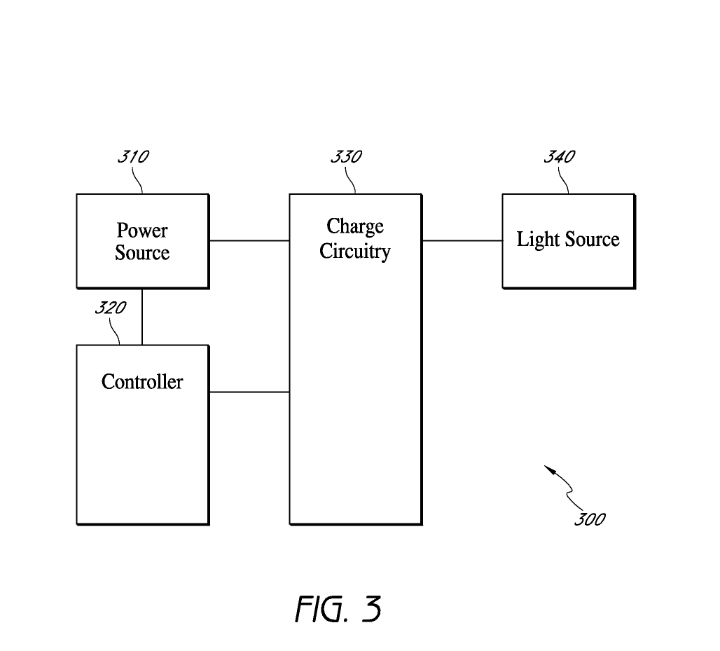

In the illustrated embodiment, the device 100 includes a housing 110 that contains the device's internal components. The mechanical and electronic parts used to operate the device 100 are contained within the housing 110. In the illustrated embodiment, a light source 120, a rapid flash unit 130, a processor 140 (sometimes referred to as a controller 140), a user input interface 150, a safety system 160, and an output interface 170 are carried by or contained within the housing 110 of the device 100. In various embodiments, the functional blocks 110-160 may be arranged in a manner generally different from that shown in FIG. 1. In some embodiments, for example, and as described below with respect to FIGS. 3-5 below, the rapid flash unit may include a processor and a light source such as the processor 140 and the light source 120. In other embodiments, the rapid flash unit 130 may include either a processor 140 or a light source 120.

In some embodiments, light generated by the light source 120 and emitted from the device 100 has a wavelength configured or selected to penetrate the outer layers of the skin sufficiently cause a photo-dynamic effect that kills the P. Acnes bacteria and thereby treats acne. P. Acnes bacteria can be one cause of acne. By destroying the bacteria, the device 100 removes acne blemishes from a user's skin.

In some embodiments, light emitted from the device 100 penetrates the outer layers of the skin causing a thermal effect that kills the P. Acnes bacteria. In some embodiments, the photo-dynamic effect is the primary effect that leads to killing the P. Acnes bacteria. In other embodiments, the thermal effect is the primary effect. In yet other embodiments, the photo-dynamic and thermal effect are relatively equal.

Embodiments of the device 100 include a first energy source (e.g., the light source 120) and a second energy source. In certain embodiments, the second energy source may include a heat source (not shown) such as one of the heat sources described herein (e.g., the heat sources described with respect to FIGS. 19-24B). As described in greater detail below with respect to FIGS. 19-24B, for example, in some embodiments, the heat source may be activated to provide heat to an output window, the heated window is placed in contact with a treatment area, and light is transmitted through the window to the treatment area. The device 100 may also include one or more other energy sources in certain embodiments. In addition, the energy sources may provide therapeutically significant amounts of various forms of energy including optical, electrical, thermal, or some other form of energy. One or more of the energy sources may deliver more than one form of energy to the treatment area. For example, as discussed, the light source 120 may deliver optical energy having a photo-dynamic effect and/or thermal energy to the treatment area.

The light emitting device 100 generally emits optical energy having a therapeutically significant effect and delivers the light to a treatment area. The operation of the device 100 is controlled by the processor 140. The light source 100 generates and emits the light, and may be, in one embodiment, a flash lamp. The rapid flash unit 130 powers the light source 120. In an embodiment, the rapid flash unit 130 rapidly charges a storage element, such as a battery. The rapid charging, in some embodiments, allows the device 100 flash the light source 120 enough times over a treatment period to deliver a therapeutically significant amount of optical energy to the treatment area. The output interface 170 generally provides an interface for contacting the device 100 with a user's skin. The safety system 160 operates to ensure safe operation of the device. For example, the safety system 160 prevents operation of the device 100 under certain circumstances, such as when the device 100 is not in contact with the user's skin. The user input interface 150 provides the interface for the user to activate and generally use the device 100. For example, the user input interface 150 may include a button for activating the device 100.

The rapid flash unit 130 provides the power to operate the device 100. In some embodiments, the rapid flash unit 130 allows the device 100 to rapidly flash the light source 120 (not shown, described below). The rapid flash unit 130 is capable of rapidly flashing the light source 120. The multiple flashes are emitted advantageously in a relatively short period of time, providing higher optical energy at a treatment area during a treatment period. Without this capability, energy may be lost or dissipated in between flashes and a sufficient amount of energy may not be delivered to the treatment area in order to treat the acne blemishes. The rapid flashing of the device 100 helps to maintain a therapeutically significant amount of energy at a treatment area during a treatment period.

In another embodiment, the rapid flash unit 130 delivers optical energy such that a therapeutically significant temperature is reached at the treatment area during a treatment period. For example, in various embodiments, the rapid flash unit 130 reaches a temperature from between about 40 and about 60 degrees Celsius at the treatment area during a treatment period. In one embodiment, the rapid flash unit 130 is capable of reaching a temperature of between about 47 degrees Celsius and 51 degrees Celsius at the treatment area. For example, in one embodiment, the rapid flash unit 130 reaches a temperature of about 49 degrees Celsius at the end of a treatment period. In another embodiment, the rapid flash unit 130 reaches a temperature of at least 49 degrees.

In various embodiments, the device 100 reaches the therapeutically significant temperature at the treatment area in a relatively short period of time. For example, in one embodiment, the rapid flash 130 allows the device 100 to reaches the therapeutically significant temperature in less than 5 seconds. In another embodiment, the therapeutically significant temperature is reached in less than 2 seconds. In various embodiments, the therapeutically significant temperature is reached in about 1, 2, and 5 seconds. As described, a therapeutically significant temperature can be reached by controlling the light source with the rapid flash unit. In certain embodiments, the therapeutically significant temperature can be reached by using the heat source instead of, or in addition to, using the light source and rapid flash unit. Moreover, the heat source may operate in accordance with methods described with respect to FIGS. 13-17).

In another embodiment, the rapid flash unit 130 delivers optical energy such that a therapeutically significant average energy density is reached at the treatment area during the treatment period. In various embodiments, the rapid flash unit 130 is capable of reaching an average energy density of from between about 1 J/cm² and about 10 J/cm² at the treatment site during a treatment cycle. In another embodiment, the rapid flash unit 130 is capable of reaching an energy density in the range from about 1 J/cm² and about 3 J/cm². In one embodiment, the energy density is about 6 J/cm².

As described, the device may be flashed multiple times over the treatment period. In various embodiments, for example, the number of flashes may be 1, 2, 4, 6, or 10 flashes. In various embodiments, the rapid flash unit 130 allows the device to flash the light source within a relatively short period of time. In one embodiment, for example, the rapid flash unit 130 allows the device to flash the light source in 333 milliseconds. For example, in some embodiments, the rapid flash unit 130 allows the device to flash the light source within 500 milliseconds of a previous flash. In other embodiments, the rapid flash unit 130 allows the device to flash the light source within less than 500 milliseconds of a previous flash, for example, within 100 milliseconds. In one embodiment, the rapid flash unit 130 allows the device to reach a temperature of about 49 degrees Celsius at the treatment area in about two seconds in six flashes or less of the light source 120.

In various embodiments, the rapid flash unit 130 allows the device to flash the light source 120 at a relatively fast frequency. For example, in various embodiments, the rapid flash unit 130 enables the device 100 to flash the light source 120 at a frequency of up to about 10 Hz. In various embodiments the rapid flash unit 130 may flash the light source 120 at frequencies of about 1, 5, and 10 Hz. In another embodiment, the rapid flash unit 130 may flash the light source 120 at about 2 Hz. For example, in one embodiment, the rapid flash unit 130 allows the device to reach a temperature of about 49 degrees Celsius at the treatment area in three seconds with six evenly spaced flashes, corresponding to a flashing frequency of 2 Hz.

In some embodiments, the rapid flash unit 130 includes a power source such as a battery (such as a disposable or a rechargeable battery), a power cell, a fuel cell, and/or a capacitor. The power source may be located in the rapid flash unit 130, or in another part of the device 100. In other embodiments, the rapid flash unit 130 includes a rechargeable power source, such as a lead and sulfuric acid, nickel cadmium (NiCd), nickel metal hydride (NiMH), lithium ion (Li-ion), or a lithium ion polymer (Li-ion polymer) battery). The device of some embodiments includes an input and associated circuitry which allows the device to be charged via an AC adapter. In one embodiment, the power source is disposable, such as a disposable battery. In some embodiments, the rapid flash unit 130 includes a capacitor which is charged by the power source. In some embodiments, the rapid flash unit includes a single capacitor capable of holding the entire charge needed to power the device 100. In other embodiments, multiple, smaller capacitors are used. The rapid flash unit 130 generally includes capacitor charging, control, and light source triggering circuitry as well. Embodiments of such circuits are discussed below with respect to FIGS. 3-5.

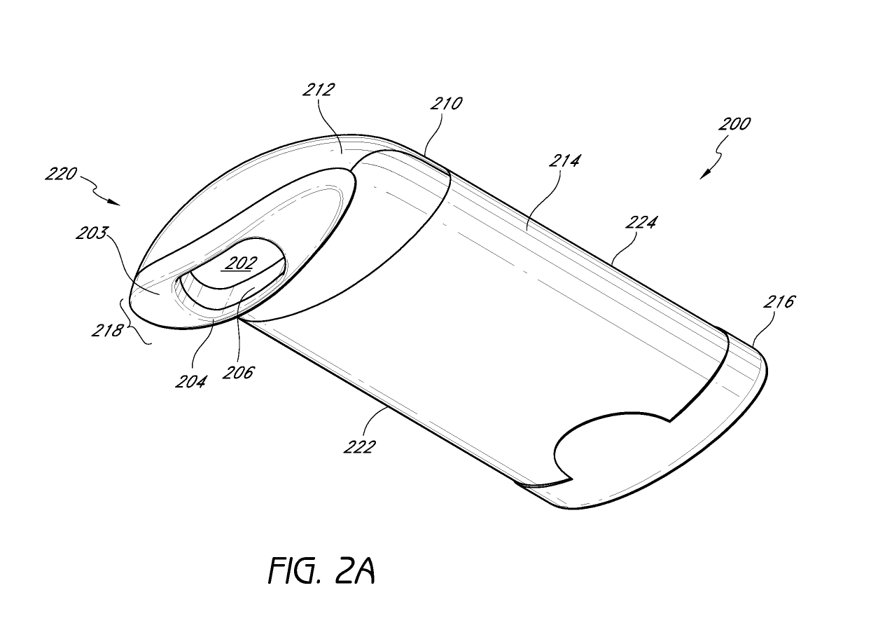

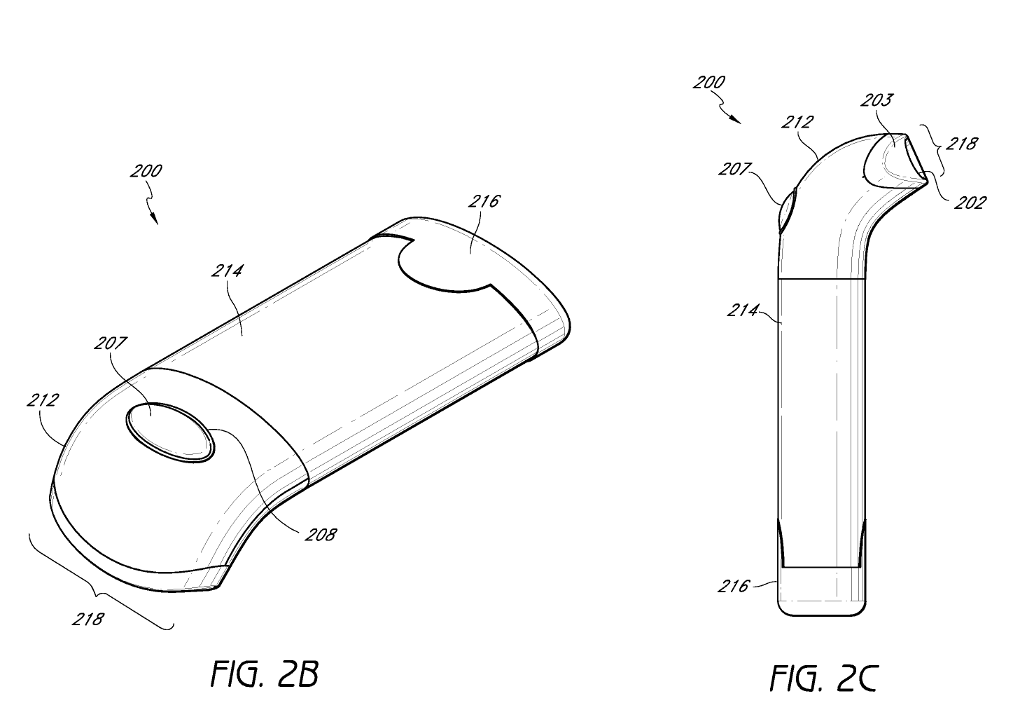

FIGS. 2A-C illustrate another embodiment of the device 100. In some embodiments, the device 200 is the same as, or includes one or more of the same components of the device 100 described above with respect to FIG. 1. The device 200 of certain embodiments includes a rapid flash unit (not shown), which in some embodiments is the same as the rapid flash unit 130 described above with respect to FIG. 1.

The rapid flash unit may be physically located in any portion of the device 200. Components of the rapid flash unit may be physically separated within the device. For example, in one embodiment, the battery or power source may be in a different portion of the device than the charging, control, and light source triggering circuitry. For example, in one embodiment, the rapid flash unit is located in the base portion 216, which advantageously provides a counterbalance to the weight of the output interface 218 and provides easy access to a user. Various embodiments of a rapid flash unit are discussed below.

The device 200 includes a housing 210, which is configured to hold device components. For example, the housing 210 can hold a light source, power source, controller, user interface, safety system, and/or output interface. The housing 210 includes a head portion 212, a body portion 214, and a base portion 216. An output interface 218 (sometimes referred to as a user interface 218), is coupled to the housing 210 at the device's light-emitting end 220. The housing of certain embodiments also includes a heat source such as any of the heat sources described below with respect to FIGS. 18-24B. The housing 210 may optionally also include a temperature sensor and/or a cooler, such as those describe below. In some embodiments, the heat source, sensor, and/or cooling device are located within the output interface 218. In other embodiments, these components are located within other portions of the housing 210. In one embodiment, the portions 212, 214, 216 are removably attachable to one another. In other embodiments, the housing 210 is a single, molded, contiguous piece.

In other embodiments, the output interface 218, or portions thereof, are removably attachable to the housing 210. The removably attachable portions may be removed by using any of a variety of mechanisms, including friction mechanisms, such as friction locks, snaps, sliders, ridges, threads, etc., as well as other mechanical devices, including screws, locks, rings, etc.

The various segments or portions thereof that are removably attachable may be disposable. For example, the entire head portion 212, output interface 218, or portions thereof are disposable in some embodiments.

The housing 210 is ergonomically shaped and helps avoid fatigue during use. In some embodiments, for example the body portion 214 forms a handle region large enough accommodate a user's hand comfortably while allowing a firm grip. Moreover, in the illustrated embodiment, the sides 222, 224 of the device 200 are rounded to comfortably accommodate the users thumb and fingers when gripping the device 200.

The housing 210 may be made of, for example, various types of metal, plastic, rubber, or a combination thereof In some embodiments, the segments 212, 214, 216 of the housing 210 are made from different materials. For example, in some embodiments, the head portion 212 is made from plastic and the body portion 214 is made of metal, or vice versa.

In one embodiment, the device 200 includes a controller (not shown), such as the processor 140 described above with respect to FIG. 1 or the processor 1840 described below with respect to FIG. 18. In one embodiment, the controller (or processor 140 or processor 1840) is made from discrete logic only, and does not include a microprocessor or microcontroller. In such embodiments, the device 200 does not include any software or firmware. This advantageously helps simplify the electronics, reduces costs, and can greatly simply design validation as well as regulatory review by agencies such as the Food & Drug Administration (the FDA).

In other embodiments, the controller (or processor 140) includes a controller, microcontroller, or memory, including a PIC microcontroller, embedded logic, a ROM, an EPROM, an EEPROM, a field-programmable gate array (FPGA), firmware or other programmable logic device (PLD). In other embodiments the controller (or processor 140) includes and ASIC, a soft microprocessor, or a complex programmable logic device (CPLD).

The controller controls operation of the device 200, as discussed in greater detail below. In general, wherever operation of the device 200 is discussed below, the controller may be the component which implements the operation even if not specifically mentioned with respect to the described operation.

In various embodiments, the controller includes a general purpose, single-chip or multi-chip microprocessor (such as a Pentium® processor, a Pentium® II processor, a Pentium® Pro processor, an xx86 processor, an 8051 processor, a MIPS® processor, a Power PC® processor, or an ALPHA® processor). In addition, the controller may include a special purpose microprocessor, such as a digital signal processor.

The device 200 also includes a power source (not shown), which in some embodiments is the same as the rapid flash unit 130 described above with respect to FIG. 1 or the power source 1830 described below with respect to FIG. 1800. The power source provides the power to operate the device 200. In some embodiments, the power source includes a battery (such as a disposable or a rechargeable battery), a power cell, a fuel cell, and/or a capacitor. In some embodiments, the power source includes a single capacitor capable of holding the entire charge needed to power the device 200. In other embodiments, multiple, smaller capacitors are used. As described above, the power source 130 may be included in a rapid flash unit such as one of the rapid flash units described in the '933 application. For example, the power source 130 may be one of the power sources (e.g., one of the one or more capacitors) described with respect to FIGS. 3-5 of the '933 Application and which are incorporated by reference herein. The power source generally includes capacitor charging, control and light source triggering circuitry, as well. The control, charging and light source triggering circuitry may also be the control, charging and light source triggering circuitry described with respect to FIGS. 3-5 of the '933 Application. Embodiments of such circuitry are incorporated by reference herein.

The power source may be physically located in any portion of the device 200. For example, in one embodiment, the power source is located in the base portion 216, which advantageously provides a counterbalance to the weight of the output interface 218 and provides easy access to a user. In one embodiment, the power source is disposable, such as a disposable battery. In other embodiments, the power source 130 is rechargeable, such as a lead and sulfuric acid, nickel cadmium (NiCd), nickel metal hydride (NiMH), lithium ion (Li-ion), or a lithium ion polymer (Li-ion polymer) battery).

The device 200 also includes a light source (not shown), which in some embodiments is the same as the light source 120 described above with respect to FIG. 1 or the light source 1820 described below with respect to FIG. 1800. In some embodiments, the light source is configured to emit a broad spectrum light. For example, the light source can include a flashlamp, such as a xenon or krypton gas filled flashlamp, or other broadband light source. Broadband light sources can be configured to emit light having wavelengths in the range of from about 400 nm to about 1100 nm in certain embodiments. In other embodiments, for example, broadband light sources can be configured to emit light having wavelengths in the range of from about 400 nm to about 1200 nm.

In other embodiments, the light source is configured to emit monochromatic or substantially monochromatic light. For example, the light source can include an LED, diode, laser, or other narrow-band light source. In yet other embodiments, broad spectrum and monochromatic light is combined from one or more light sources, or alternated in their application from the device 200. In other embodiments, light of multiple wavelengths is emitted simultaneously or sequentially.

The output from the light source can be controlled or modulated prior to delivery to the user. For example, in some embodiments, the light emitted from the light source is passed through a filter. In one embodiment, the filtered light has a wavelength greater than 400 nm. The filtered light can have wavelengths in a range from about 400 nm to about 1100 nm, or from about 400 nm to about 700 nm. In some embodiments, the filtered light can have wavelengths in a range from about 400 nm to about 1200 nm. In other embodiments, the filtered light has a wavelength mostly at around 400 nm. The filter can be provided as an optical coating to the light source (e.g., flashlamp), or as a window positioned between the light source and the user's treatment site.

In addition, the optical characteristics of the light generated by the light source can be controlled by the controller, or other electronic circuitry included with the housing 210. For example, by pulsing the light source 120, the pulse shape of the emitted light can be modulated and controlled. In addition, by varying the drive current and/or voltage to the light source, the output power can be modulated or controlled.

In one embodiment, light emitted from the light source has a wavelength of about 400 nm. In other embodiments, the wavelength is greater than 400 nm. For example, in some embodiments, the wavelength is between about 400 and 700 nm. In other embodiments, the wavelength may be between 400 and 1100 nm. In some embodiments, the wavelength may be greater than 1100 nm. In yet other embodiments, the wavelength may be greater than 1200 nm. In one embodiment, the wavelength is in the blue spectrum, and the light emitted from the light source is blue light.

As discussed above, the particular wavelength or range of wavelengths directed to a treatment site on a user can be controlled by using a narrow band light source configured to emit light at a desired wavelength (or range of wavelengths), or by using a broad band light source with filters to filter out or remove undesired light wavelengths.

In some embodiments, the light source includes both a broadband light source and a narrowband light source. For example, in one embodiment, the light source 120 includes a flashlamps and one or more light emitting diodes (LEDs).

In another embodiment, the light source includes two flash lamps, each having a different optical coating configured to filter out different wavelengths. For example, in one embodiment, one coating is designed to transmit light in the infrared spectrum (or a portion thereof), and the other coating is designed to transmit light corresponding to the visible blue spectrum (or a portion thereof). In another embodiment, the two flashlamps are each housed in a different chamber within the housing, each chamber having a different filter window at its chamber output. In other embodiments, only one primary wavelength of light is transmitted through the optical coating, while in other embodiments, light having multiple primary wavelengths is transmitted therethrough.

In some embodiments, the wavelength or wavelengths transmitted to a treatment site are selectable; either automatically by the device 200 itself, or by the user via a user interface (not shown). For example, in some embodiments, a user can select a desired treatment wavelength from a range using, for example, a series of buttons or a dial corresponding to a variety of wavelength ranges. Alternatively, the device 200 can include a digital user interface which allows a user to select a wavelength range or ranges from a menu displayed on a display. In some embodiments, the user selection will cause different types of optical filters to be placed in the path of the light source's output. In some embodiments, for example, a dial is mechanically coupled to an optical filter having different filtering materials on different sections, and turning the dial causes the filter material placed in the light path to change. In other embodiments, user selection causes the controller to actuate a motor or other device to move the desired filter into place.

The optical characteristic of the light emitted by light source may be varied as a function of skin pigmentation. For example, the variation may be based on the Fitzpatrick Classification Scale of skin pigmentation types. For example, when the user indicates that the device 200 is going to be used to treat a darker skin types, the controller will control the light source to generate pulsed optical energy having a longer pulse duration than when lighter skin type treatment is selected.

The optical characteristic may be automatically selected by the controller based on a user selection of skin pigmentation type, or by sensing the treatment site skin pigmentation. For example, the device 200 may include a dial or other form of user input interface to allow a user to set their skin pigmentation type. The controller could then, based on the user's skin pigmentation type, select the appropriate treatment pulse duration, peak power, average power, pulse interval, duty cycle, etc., and would actuate the flash lamp accordingly. In one embodiment, the number of pulses in a treatment cycle may be changed based on the user's skin pigmentation.

In other embodiments, the user selects the optical characteristic directly instead of selecting their pigmentation type and using the controller to determine optical characteristic. In yet other embodiments, the device 200 includes a colorimeter or other device to automatically determine the pigmentation of a user's treatment site. Furthermore, light emitted from the light source is sometimes characterized as an intense pulse of light. In some embodiments, the light source is removable from the device 200 by the user, either by hand, or with a tool.

In some embodiments, the light source includes a reflector to reflect, direct, and/or focus energy emitted from the light source towards the patient's skin. The reflector increases the amount of light received by the tissue and thereby increases the photo-therapeutic effect. In some embodiments the reflector has a parabolic cross sectional shape and extends along substantially the entire length of the light source. In other embodiments the reflector has a concave cross section.

The acne treatment device 200 also includes a user input 207 (which in some embodiments is the same as the user input interface 150 described above with respect to FIG. 1). The user input 207 for user control of various operational features. For example, in various embodiments, the user input 207 includes a switch, button (as illustrated), contact, and/or sensor. In some embodiments, the user input 207 causes the device 200 to turn on and/or off, to charge a power supply, to begin or end light flashing, to program the exposure duration, and/or to enter a code to re-activate the device 200. Embodiments of a user input are described in greater detail below.

As discussed below, the rapid flash unit, the user input, safety system, and output interfaces operate together to provide various clinical and safety advantages. For example, the rapid flash unit allows the device to rapidly flash light of sufficient energy to treat various dermatological conditions, such as killing or reducing Acne Vulgaris. However, by providing a safety system with the rapid flash unit, the user is able to make sure that energy is emitted from the device 200 only when it is safe to do so. For example, in one embodiment, the device will not permit light activation unless the safety system determines that the device is in contact with the user's skin.

FIG. 3 is a block diagram of a rapid flash unit 300 of an acne treatment device according to another embodiment of the disclosure. In one embodiment, the rapid flash unit 300 includes one or more of the same components of the rapid flash unit 130 of the device 100 described above with respect to FIG. 1 and/or the rapid flash unit of the device 200 described above with respect to FIG. 2. In the illustrated embodiment, the rapid flash unit 300 generally includes a power source 310, a controller 320, charge circuitry 330, and a lamp 340. The rapid flash unit 300 generally allows the light source of the device to flash in rapid succession in order to maintain a therapeutically significant amount of energy at a treatment site, or to raise and maintain the temperature at a treatment site to a predetermined therapeutic level during a treatment period.

In one embodiment, the power source 310 may power the entire device. In another embodiment, the power source 310 be one of multiple power sources and may power only a portion of the device, such as, for example, the components of the rapid flash unit 300. The power source may, in one embodiment, for example, provide a DC voltage level to the controller 320 and the charge circuitry 330 of the rapid flash unit 300.

In one embodiment, the controller 320 controls the charge circuitry 330 and the lamp 340. The controller 320 may include the processor 140 described above with respect to FIG. 1 and/or the processor described above with respect to FIG. 2. Alternatively, the controller 320 may include a separate controller. For example, the controller 320 may only control components of the rapid flash unit 300 and not the entire device. Alternatively, in other embodiments, the controller 320 may control the entire device or other portions of the device in addition to the rapid flash unit 300.

In one embodiment, the controller 320 is made from discrete logic only, and does not include a microprocessor or microcontroller. In other embodiments, the controller 320 includes a controller, microcontroller, or memory, including a PIC microcontroller, embedded logic, a ROM, an EPROM, an EEPROM, a field-programmable gate array (FPGA), firmware or other programmable logic device (PLD). In other embodiments the controller 320 includes and ASIC, a soft microprocessor, or a complex programmable logic device (CPLD).

The charge circuitry 330 generally charges an energy storage element sometimes referred to as an energy storage device. Charge stored with the storage element is then used to activate or flash the light source 340. The light source 340 may be any of the light sources described above. The charge circuitry may include discrete devices only or may be implemented in part by a micro-controller. In one embodiment, the storage element includes one or more capacitors.

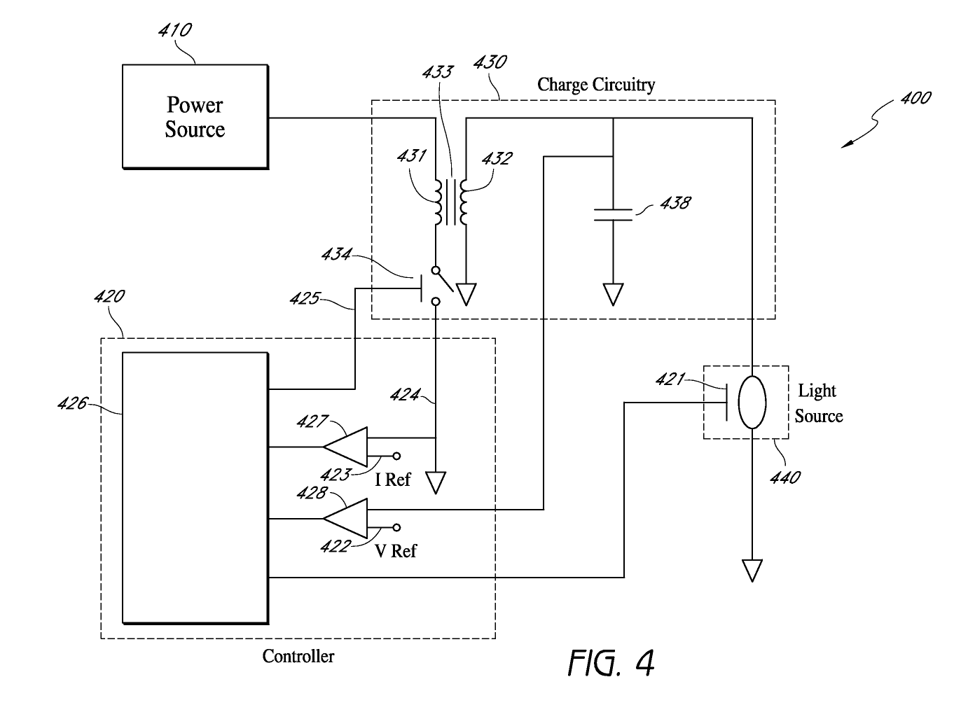

FIG. 4 is a functional schematic diagram of a rapid flash unit 400 of an acne treatment device according to yet another embodiment. The rapid charge includes one or more of the same components of the rapid flash unit 130 of the device 100 described above with respect to FIG. 1, the rapid flash unit of the device 200 described above with respect to FIG. 2, and/or the rapid flash unit 300 described above with respect to FIG. 3. In some embodiments, the rapid flash unit 400 is a switching power supply which is used to generate the voltage used to flash or activate the light source 440. For example, in one embodiment, the voltage need to flash the light is relatively high (e.g., 300V). The rapid flash unit can generate this relatively high voltage from a relatively smaller voltage that is provided by the battery or power source (e.g., 12V).

In one embodiment, the rapid flash unit 400 includes a controller 420, which includes a micro-controller 426, a current monitor 427, and a voltage monitor 428. The rapid flash unit further includes a transformer 433, an energy storage element (or energy storage device) 438, and a triggerable light source 440.

In one embodiment, current flowing through the primary coil 431 of the transformer 433 increases generally linearly as the transformer's 433 core beings to saturate. When the current hits a threshold level, the controller 420 detects the saturation with the current monitor 427, and ceases delivering current to the transformer 433. The threshold current level corresponds to saturation of the magnetic flux in the transformer's 433 core. The controller 420 determines when the threshold current level is reached using the current monitor 427. In certain embodiments, the transformer saturation point is from between about 3 Amps and 10 Amps. In one embodiment, for example, the transformer saturation point is about 5 Amps. In yet other embodiments, the saturation point is another value compatible with embodiments described herein and may be less than 3 Amps or greater than 10 Amps, for example.

When the transformer's primary 431 ceases receiving current, the magnetic core collapses, and discharges current through the transformer's secondary winding 432. The current pulse is fed to the energy storage element 438. The current pulse deposits a partial charge to the storage element 438. The voltage monitor 428 monitors the total stored energy or voltage across the storage element 438. If the voltage monitor 428 determines that the threshold voltage has not yet been reached (e.g., the storage element 438 is not adequately charged), the voltage monitor 428 signals the controller 420 to repeat the process of sending current to the transformer's primary coil 431, as discussed above.

If the voltage monitor 428 determines that the threshold voltage across the storage element 438 has been reached, the voltage monitor 428 signals the controller to trigger the light source 440. In one embodiment, the controller 420 sends a trigger signal to a trigger 421, ionizes the gas in the light source 440. When the gas ionizes, the light source's resistance drops sufficiently for the energy stored in the energy storage element 438 to discharge across the light source 440. As the energy storage element 438 discharges, the light source 440 emits a bright flash of light.

This process is repeated a number of times, until a therapeutically significant amount of energy has been generated by the light source.

Advantageously, in one embodiment, for each pulse of current delivered to the transformer's 433 primary coil 431, the rapid flash unit 400 allows the magnetic flux in the transformer's 433 core to saturate (or reach a point near saturation) by supplying one continuous pulse of current. As a result, the corresponding pulses of charge delivered on the transformer's secondary coil 432 are relatively large and delivered relatively quickly to the storage element 438. This reduces the overall energy stored in device 438 (e.g., a capacitor) charging time. Alternatively, in another embodiment, the rapid flash unit 400 delivers multiple pulses of current in order to gradually saturate the core of the transformer 433. In one embodiment, the multiple pulses generally increase in duration as the transformer's 433 core approaches saturation. In yet another embodiment, the magnetic field of the transformer's 433 core is allowed to collapse by ceasing delivery of current to the primary 431 before the transformer 433 reaches a point at or near saturation.

In addition, in one embodiment, the rapid flash unit 400 charges the storage element 438 by depositing charges of generally equal magnitude at a generally constant frequency throughout the entire charging period. This allows the rapid flash unit 400 to reduce the amount of time required to reach the threshold voltage. Alternatively, in another embodiment, the magnitude of the individual pulses of charge vary throughout the charging period. In one embodiment, for example, the magnitude of the charges generally decreases as the total charge across the storage element approaches the threshold voltage. In another embodiment, the period of time between individual pulses of charge changes over the charging period. For example, the period of time between individual pulses of charge may generally increase as the charge across the storage element approaches the threshold voltage.

As discussed generally above, the rapid flash unit 400 generally operates by supplying switched current at a relatively high frequency to the primary winding 431 of the transformer 433. For example, in certain embodiments, the rapid flash unit 400 switches the current from between about 20 KHz and about 80 KHz. In one embodiment, the rapid flash unit switches the current at less than 20 KHz, at 10 KHz, for example. In another embodiment, the current is switched at greater than 80 KHz, at 100 KHz, for example. In one embodiment, the rapid flash unit switches the current at 20 KHz and the capacitor 438 is charged to 300 volts using about 10,000 pulses of 300 mill volt charge over a 500 millisecond charging period. The power source 410 is connected to and provides a voltage to a first terminal on the primary winding 431 of a transformer 433. The second terminal of the primary winding 431 is connected to a switch 434, controlled by controller 420 using a control signal 425. When the switch 434 is closed, current flows through the primary winding 431. The first terminal of the secondary winding 432 of the transformer 433 is connected to an energy storage device 438, such a capacitor. One terminal of the light source 440 is also positioned near the trigger 421. The light source 440 is also connected to the controller 420 via a control signal 421, which is used to flash the light source 440 as described above. A current monitor 427 compares a reference input 423 to the current flowing through the wire 424 corresponding to the current flowing through the primary winding 431. The output of the current monitor 427 is directed to the controller 426 (e.g. a micro-processor 426). A voltage monitor 428 compares the voltage on the energy storage device 438 to a reference voltage provided at a reference voltage terminal 422. The output of the voltage monitor 428 is provided to controller 426.

The switched current is provided to the transformer 433 by opening and closing the switch 434. Opening and closing the switch turns off and on, the current through the primary winding 431 of the transformer 433. The controller 420 initially closes the switch 434, causing current to flow through the primary winding 431 of the transformer 433, respectively. When the controller 420 detects, using the current monitor 427, that the primary winding of the transformer 433 is at or near the point of saturation, the controller 420 opens the switch 434, causing current to stop flowing through the primary winding 431. As a result, the magnetic field in transformer 433 collapses, inducing a change in voltage and corresponding change in current on the secondary winding 432 of the transformer 433, and partially charging the capacitor 438. The controller 426 determines whether the capacitor 438 is charged sufficiently to flash the light source 440 by comparing the charge on the capacitor 438 to a reference voltage 422 using the voltage monitor 428. If the capacitor 438 is not charged sufficiently to flash the light source 440, another pulse of charge is supplied to the capacitor 438 by repeating the process of opening and closing the switch 434. Once the controller 426 detects that the capacitor 438 has been sufficiently charged, the controller 426 will flash the light source 440 by activating the trigger 421.

In some embodiments, the transformer 433 may advantageously be a fly-back transformer (also referred to as a line output transformer). In other embodiments, the transformer 433 may be another type of transformer, such as a forward boost converter. The switch 434 may be a transistor, such as, for example, a field effect transistor (FET). In other embodiments, the switch may be another type of transistor or switching device. In some embodiments, the energy storage device 438 includes multiple capacitors rather than a single capacitor. The capacitor 438 may be a single capacitor sufficient to store the entire charge required to flash the light source 441 or may include a bank of capacitors. The light source 440 may be any of the light sources described above or may be another light source.

Embodiments of the rapid flash unit 400 may include additional components not shown in FIG. 4. For example, various other circuit components (e.g., resistors, capacitors, etc.) may be included to implement the various functional blocks of flash unit 400.

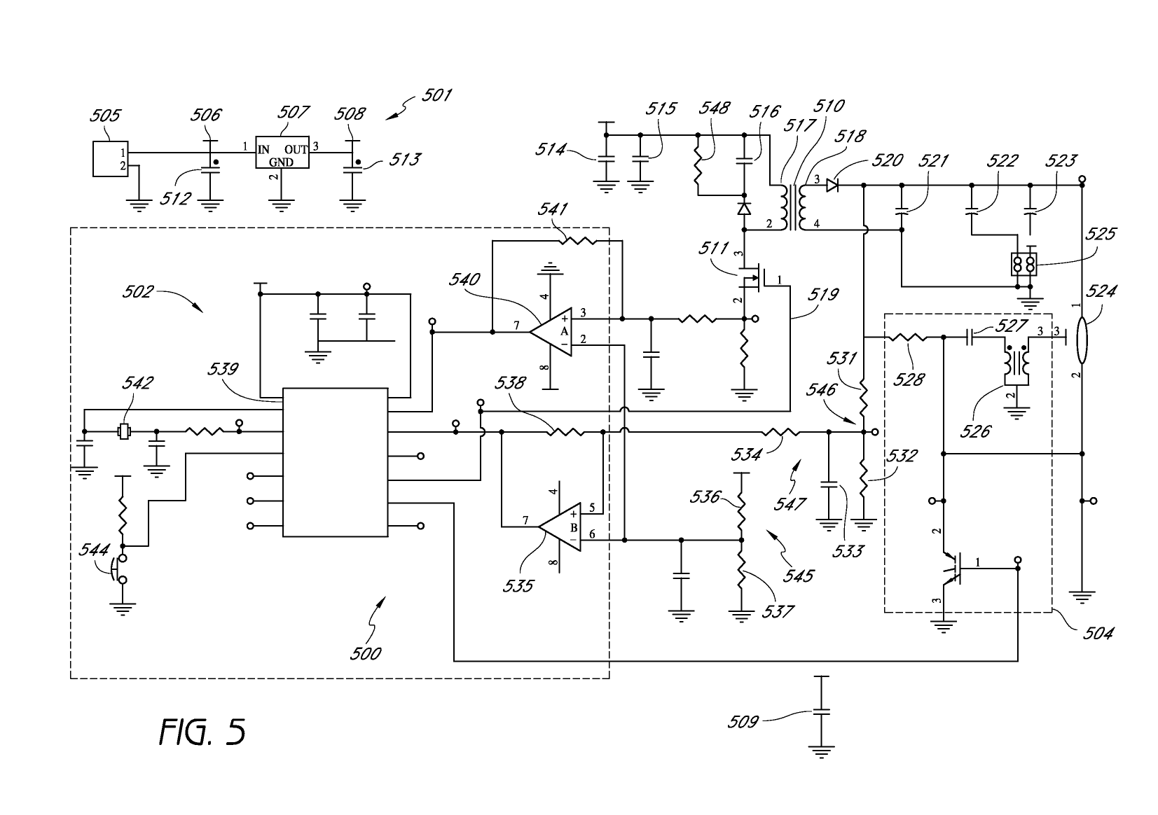

FIG. 5 is a schematic diagram of a rapid flash unit 500 of an acne treatment device according to one embodiment of the disclosure. In one embodiment, the rapid flash unit 500 includes one or more of the same components of or is the same as the rapid flash unit 130 of the device 100 described above with respect to FIG. 1, the rapid flash unit of the device 200 described above with respect to FIG. 2, and/or the rapid flash units 300, 400 described above with respect to FIGS. 3 and 4, respectively.

The rapid flash unit 500 includes a power supply portion 501, a control portion, charge circuitry, and a light source with associated triggering circuitry 504. The power supply 501 includes a 12 volt supply 506. The 12 volt supply 506 is connected to a voltage regulator 507 which provides a 5 volt supply 506 to certain components of the rapid flash unit 500. The power supply 501 includes several filtering capacitors 509, 512, 513. In other embodiments, the power supply 501 may include other supply values and may include other support circuitry as is known in the art.

The charge circuitry 503 includes a fly-back transformer 510 having a primary coil 517 and a secondary coil 518. The 12 volt supply 506 which can be an un-regulated supply, is connected to a first terminal of the primary coil 517 as well as several capacitors 514, 515, 516 and a resistor 548. A first terminal of a FET 511 is connected to the second terminal of the primary coil 517. Control line 519 is coupled to a second terminal of FET 511 and is used to turn on or actuate the FET 511. When actuated, current flows from the supply 501, through the primary coil 517, and then through the FET 511.

The magnitude of the current through the primary coil 517 is compared to a reference with a comparator 540. The reference value is set by a voltage divider 545, which includes resistors 536, 537. The comparator 540 is, for example, a low power, low offset, voltage dual comparator. The output of the voltage comparator 540 is provided to the voltage comparator's 540 first input via resistor 541 to provide a hysteresis function, as is well known in the art. The output of the comparator 540 also is connected to a micro-controller 539. The output state of the comparator 540 will change when the voltage at the first terminal, which is proportional to the current through the primary coil 517 of the transformer 510, is a certain amount greater than the voltage at the reference input of the voltage comparator. The reference value is set using the voltage divider 545 through the selection of the resistors 536, and 537. The reference value is selected such that the comparator 540 output changes when the current through the primary coil 517 corresponds to transformer 510 core saturation.

The secondary coil 518 of the fly-back transformer 510 is connected, through rectification diode 520, to a group of capacitors 521, 522, 523, which are able to store a charge large enough to flash the xenon lamp 524 when triggered. The diode 520 rectifies the output from the fly-back transformer 510 to charge the capacitors 521, 522, 523. The diode 520 provides a half-wave rectification function. In other embodiments, a full-wave rectification circuit may be used. In one embodiment, a smoothing circuit may be included to smooth the rectified signal into a constant DC signal. A jumper is connected to two of the capacitors 522, 523 in order to allow for selective inclusion of the capacitors. In other embodiments, only a single capacitor is used as an energy storage device, and jumpers are not provided. The lamp 524 has triggering circuitry 504 which includes a transformer 526, capacitor 527, resistor 528, and bi-polar field effect transistor (bi-FET) 529. A control signal 530 switches the bi-FET 529, which, in conjunction with the rest of the triggering circuitry 504 triggers the lamp 524 to flash.

The capacitors 521, 522, 523 are also connected to ground through two resistors 531, 532, which act as a voltage divider 546. The output of the voltage divider 546 passes through a low pass filter element 547, which includes a capacitor 533 and a resistor 534. The low pass filter feeds into a first input terminal of the voltage comparator 535. The voltage comparator 535 is, for example, a low power, low offset, voltage dual comparator. The voltage divider 545 provides a reference voltage to the second terminal of the comparator 535. The reference voltage is compared to the voltage at the first terminal of the voltage comparator 535. The output of the voltage comparator 535 is fed back to the first input of the voltage comparator 535 through resistor 538 to provide a hysteresis function. The output of the comparator 535 also is connected to the micro-controller 539. The output state of the voltage comparator 535 will change when the voltage at the first terminal, proportional to the voltage stored on the capacitors 521, 522, 523, is a certain amount greater than the voltage at the reference input of the voltage comparator. The reference voltage is set such that the change in the comparator output will trigger when the capacitors 521, 522, 523 are charged sufficiently to flash the xenon lamp 524.

In the illustrated embodiment, the same voltage divider 545 is used to set the reference signal for both the comparator 540, used to monitor the current through the primary coil 517 of the transformer 510, and for comparator 535, used to monitor the voltage on the capacitors 521, 522, 523. This provides the advantage of reducing circuit complexity, size, and cost by advantageously using a single voltage divider for both current and voltage monitoring purposes. In other embodiments, separate voltage dividers could be used.

The controller includes a micro-controller 539 and associated circuitry. For example, the microcontroller is a PIC16C505 14-pin, 8-bit CMOS micro-controller. The micro-controller 539 is clocked by an oscillator 542 and is resettable by reset switch 544. As described above the controller 539 is configured to provide a control signal to the triggering circuitry 504 used to flash the xenon lamp. The controller 502 is also configured to provide a control signal to the FET 511 to pulse current into the transformer primary coil 517. The controller is also configured to receive output signals from the current and voltage comparators 535 and 540.

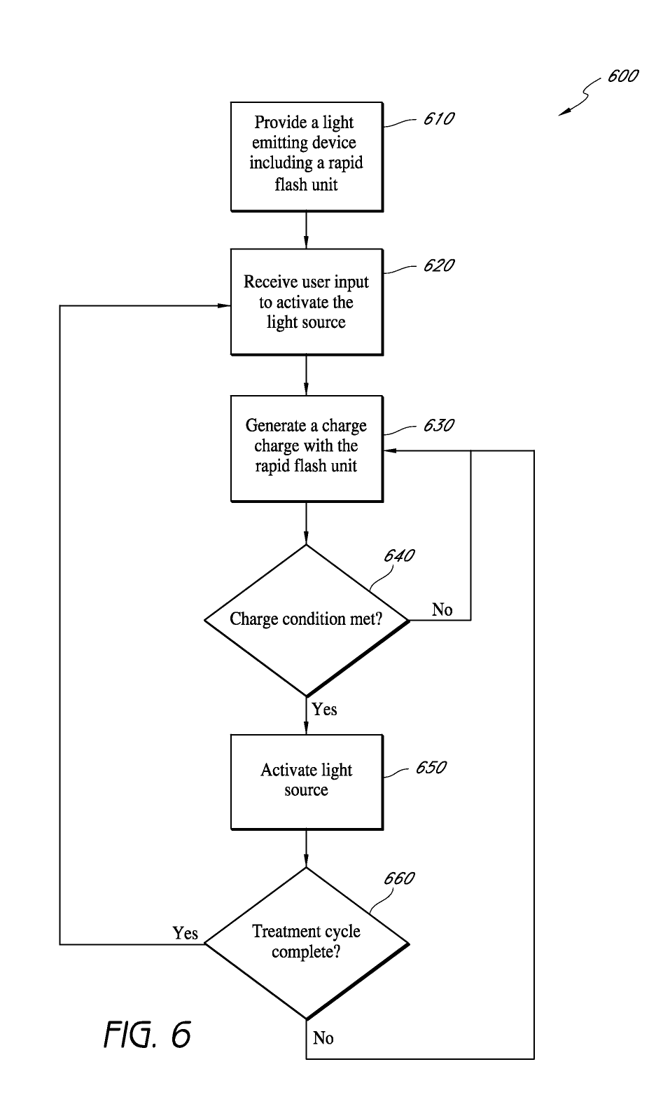

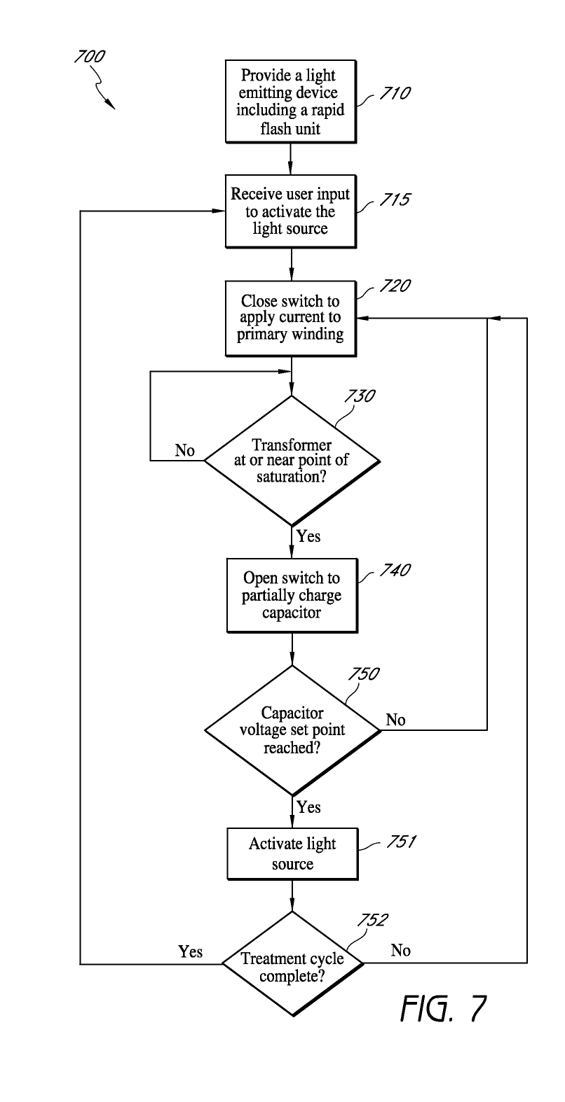

FIG. 6 shows an embodiment of a method of treating acne with an acne treatment device having a rapid flash unit, according to one embodiment of the disclosure. The method 600 begins at step 610, in which an acne treatment device is provided that includes a rapid flash unit. The light emitting device is the device 100 described above with respect to FIG. 1. In other embodiments, the light emitting device is the device 200 described above with respect to FIG. 2, or some other light emitting device. The light emitting device includes a light source configured to generate optical energy having a wavelength in a range of from about 400 nm to about 1100 nm. In some embodiments, the light source may be any of the light sources 120, above with respect to FIG. 1, the light source of FIG. 2, the light source 340 of FIG. 3, the light source 421 of FIG. 4, the light source 524 of FIG. 5, or another light source.

The rapid flash unit may be, for example, one of the rapid flash units described above with respect to FIGS. 1-5 or some other rapid flash unit. At step 620 the method 600 receives a user input to activate the light source. For example, the method 600 detects that a user has pressed a button on the device. The method 600 then generates a charge with the rapid flash unit at step 630. The charge may be generated on a capacitor using a transformer as described above, for example, with respect to FIGS. 4-5. The transformer may be a fly-back transformer as described in FIGS. 4-5. The method 600 further determines whether a charge condition is met at step 640. For example, a controller, using a voltage monitor may carry out this detection step as described above with respect to FIGS. 4-5 above. The voltage monitor may include a voltage comparator as in FIG. 5. If, at step 640, the method 600 determines that the charge condition is not met, the method 600 returns to step 630 and continues to generate a charge using the rapid flash unit. If the method 600 determines that the charge condition is met at step 640, the method 600 continues to step 650 and allows activation of the light source at step 650. At step 660, the method 600 determines whether a treatment cycle is complete. The treatment cycle may be a treatment cycle described above or another treatment cycle. For example, the treatment cycle may include a certain number of flashes. If, at step 660, the method 600 determines that the treatment cycle has not completed, the method 600 returns to step 630 and begins to generate a charge again using the rapid flash unit 630. If the method 600 determines that the treatment cycle is complete at step 660, the method 600 returns to step 620.

The method 600 may include additional steps not shown in FIG. 6. For instance, the method 600 may include activating the light source, generating the optical energy and directing the optical energy to a treatment area. The method 600 may further include filtering the optical energy prior to delivery to the treatment area. The filtering step may include removing energy having a wavelength outside of a range of from about 400 nm to about 700 nm or by removing energy having a wavelength below about 400 nm. In other embodiments, the method 800 also includes generating additional optical energy with a second light source. The additional optical energy may include mostly infrared energy.

FIG. 7 shows another embodiment of a method 700 of rapidly charging an acne treatment device. The method 700 begins at step 710, in which an acne treatment device is provided that includes a rapid flash unit. The light emitting device is the device 100 described above with respect to FIG. 1. In other embodiments, the light emitting device is the device 200 described above with respect to FIG. 2, or some other light emitting device. The light emitting device includes a light source configured to generate optical energy having a wavelength in a range of from about 400 nm to about 1100 nm. In some embodiments, the light source may be any of the light sources 120, above with respect to FIG. 1, the light source of FIG. 2, the light source 340 of FIG. 3, the light source 421 of FIG. 4, the light source 524 of FIG. 5, or another light source.

The rapid flash unit may be, for example, one of the rapid flash units described above with respect to FIGS. 1-5 or some other rapid flash unit. At step 715 the method 700 receives a user input to activate the light source. For example, the method 700 detects that a user has pressed a button on the device. The method 700 closes the switch at step 720 to apply current to the primary winding of a transformer at step 720. The switch may be, for example, the switches 434, 511 described above with respect to FIGS. 4-5, respectively. At step 730 the method 700 determines whether the transformer core is at or near a point of saturation. If the method 700 determines that the transformer is not at or near a point of saturation, the method 700 waits at step 730 while current continues to be delivered to the primary winding.

Once the method 700 determines that the transformer is at or near a point of saturation at step 730, the method 700 opens the switch at step 740. When the switch is opened at step 740, the magnetic field in transformer collapses, inducing a change in the voltage and a corresponding change in current on the secondary winding of the transformer, partially charging the capacitor. At step 750 the method 700 detects whether the capacitor has been charged sufficiently to reach a predetermined voltage set point. In one embodiment, the voltage set point is an amount sufficient to activate the light source when triggered. If the method 700 determines at step 750 that the voltage set point has not yet been reached, the method 700 returns to step 720, beginning the process of supplying another pulse of charge to the capacitor by repeating steps 720-740. If, at step 750, the method 700 determines that the voltage set point has reached, the method 700 the method 700 continues to step 751 and allows activation of the light source at step 751. At step 752, the method 700 determines whether a treatment cycle is complete. The treatment cycle may be a treatment cycle described above or another treatment cycle. For example, the treatment cycle may include a certain number of flashes. If, at step 752, the method 700 determines that the treatment cycle has not completed, the method 700 returns to step 720 and closes the switch to apply current to the primary winding. If the method 700 determines that the treatment cycle is complete at step 752, the method 700 returns to step 715. The method 700 may include additional steps not shown in FIG. 7.

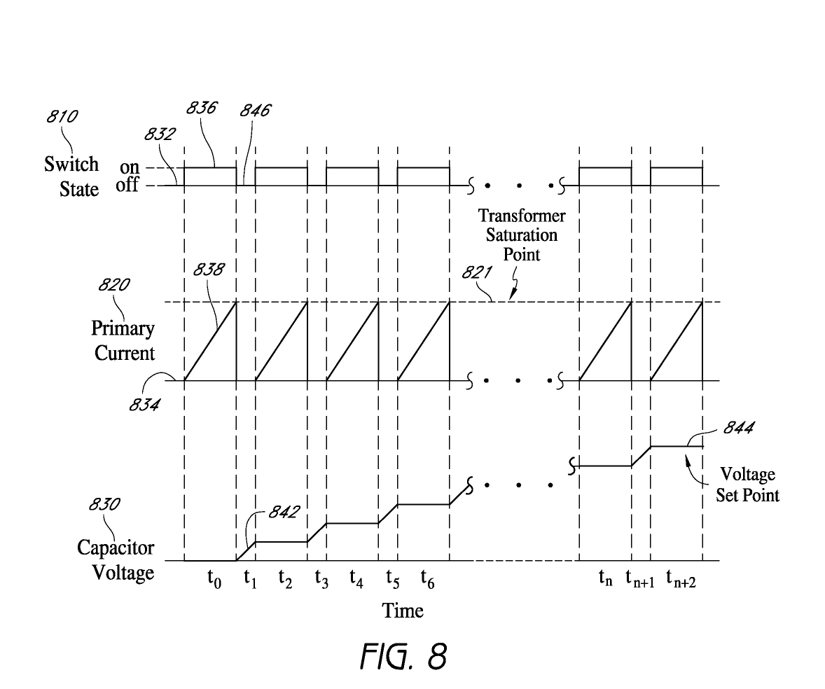

FIG. 8 shows a timing chart 800 of a rapid flash unit of an acne treatment device according to one embodiment of the disclosure. The rapid flash unit may be, for example, one of the rapid flash units described above with respect to FIGS. 1-7 or some other rapid flash unit. The chart 800 generally illustrates incrementally charging the capacitor by repeatedly pulsing current into the primary coil of the transformer. Portion 810 of the chart 800 represents the on/off state of the switch which controls the current through the primary coil of the transformer. The switch may be for example, the switch 434 described above with respect to FIG. 4. A second portion 820 of the chart 800 shows the current flowing through the primary coil of the transformer of the rapid flash unit. A third portion 830 of the chart 800 represents the amount charge stored on the capacitor which is used to flash the light source. At the beginning 832 of time period t₀, the capacitor is holding a negligible amount of charge, and the current 834 through the primary coil is negligible.

The switch is turned on (or closed) 836, causing current in the primary coil to ramp up 838 over period to until the current through the primary is large enough to indicate that the transformer has reached a point of saturation. The saturation point is shown as line 821. Next, at the beginning of time period t₁, the switch is turned off (or opened) 840. Over time period t₁ the magnetic field in the transformer collapses, causing a change in voltage and a corresponding change in current on the output of the secondary coil of the transformer. This change in the output signal of the transformer partially charges 842 the capacitor.

The above steps are repeated provide subsequent pulses of charge, providing incremental build up of charge on the capacitor. The next several pulses are shown over time periods t₁-t₆. As indicated by the broken line, the process continues until the charge on the capacitor is fully charged and reaches a voltage set point 844 at time period tₙ₊₁. At this point, the device is flashed, allowing the charge on the capacitor to deplete. In various embodiments, the number of pulses required to fully charge the capacitor will vary based on a number of factors including the amount of charge required to flash the light source, the type of transformer, the size of the capacitor, etc.

In some embodiments of the rapid flash unit, the operation of the rapid flash unit and thus the timing chart 800 may vary. For instance, in some embodiments, the rapid flash unit will not allow the charge in the capacitor to completely deplete when flashing the device. In such a case, the capacitor will not necessarily start out with a zero charge as shown (830). Moreover, the off-periods of the switch t₁, t₃, t₅ . . . tₙ₊₁ may vary in different embodiments. In some embodiments, the off-period will be long enough such that the flux in the core of the transformer is allowed to completely deplete during the off periods t₁, t₃, t₅ . . . tₙ₊₁. This mode of operation may be referred to as discontinuous mode. In other embodiments, the flux in the transformer core is not allowed to completely deplete during off time periods t₁, t₃, t₅ . . . tₙ₊₁. This mode of operation may be referred to as continuous mode and may reduce the overall charge time of the capacitor. In other embodiments, the transformer primary current will be not reach the point of saturation but will merely reach a point relatively near the point of saturation.

Portion 830 of the chart 800 shows flat segments during the on-states of the switch of the switch indicating that no charge is accruing during these time periods. During the periods when the switch is in an off-state, the portion 830 shows segments of constant, positive slope indicating that a constant ramp of charge is building on the capacitor. In some embodiments, these characteristics may be different. For example, in another embodiment, there may be some slight charge accruing when the switch is in an on-state, or the capacitor may lose some charge during these time periods. In addition, during the off-states, the capacitor may not charge at a constant rate.

As described above with respect to FIG. 4, in one embodiment, the rapid flash unit advantageously allows the magnetic flux in the transformer's 433 core to saturate (or reach a point near saturation) by supplying one, continuous pulse of current across the transformer's primary (820). This allows the rapid flash unit to deliver pulses of current to the storage element quickly, reducing the overall capacitor charging time. Alternatively, in another embodiment, the rapid flash unit delivers multiple pulses of current in order to gradually saturate the core of the transformer. In one embodiment, the multiple pulses generally increase in duration as the transformer's core approaches saturation. In yet another embodiment, the magnetic field of the transformer's core is allowed to collapse before the transformer reaches a point at or near saturation.

In addition, in one embodiment, the rapid flash unit advantageously arrives at the threshold voltage across the storage element 438 by depositing charges of equal magnitude throughout the charging period (830). This allows the rapid flash unit to reduce the amount of time required to reach the threshold voltage. Alternatively, in another embodiment, the magnitude of the individual pulses of charge vary throughout the charging period. In one embodiment, for example, the magnitude of the charges generally decreases as the total charge across the storage element approaches the threshold voltage.

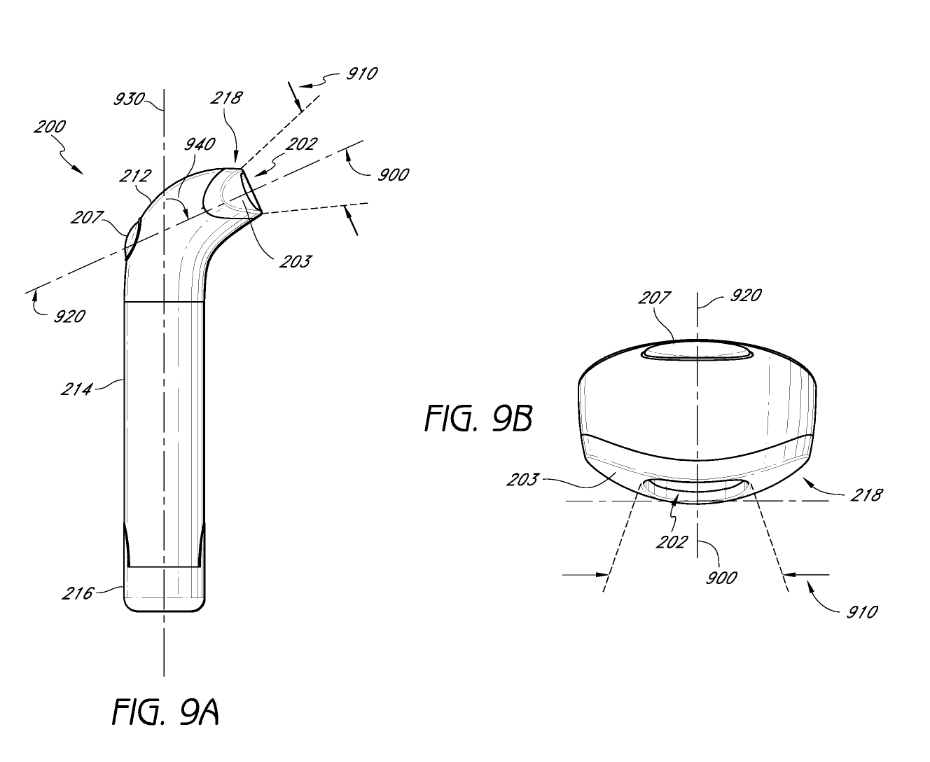

Referring now to FIGS. 9A-B, in one embodiment, light generated by the light source is configured to travel along a beam propagation axis 900 as it travels through a transmission path 202 (sometimes referred to as a transmission channel 202, emission path 202, or emission channel 202) defined by the user interface 218. The light diverges as it travels along the propagation axis 900, thereby defining a beam propagation envelope 910.

Because the light diverges as it travels along the propagation axis 900, the light's energy density decreases as the distance from the light source to the treatment site increases. Therefore, to maximize energy density, the user interface 218 of the device 200 is brought into contact with the user's treatment site prior to activating the light source. As discussed above with respect to FIG. 2, the device is configured to deliver a therapeutically significant amount of energy to the treatment site. The proper orientation of the device can help maintain the energy level at the treatment site. For example, when properly oriented, light generated by the light source will have an energy density in the range of from about 1 J/cm² to about 3 J/cm² (or from 1 J/cm² to 3 J/cm²) at the treatment site. In other embodiments, the energy density is in the range of about 1 J/cm² to about 10 J/cm² (or from 1 J/cm² to 10 J/cm²). In one embodiment, the energy density is about 6 J/cm².

In addition, in some embodiments, the light source delivers peak optical pulse power in the range of from about 5 kW to about 20 kW (or from 5 kW to 20 kW). In some embodiments, the light spot at the treatment site has an area of about 1 cm². In general, the light generated by the light source is safe to use near human eyes and will not cause serious or irreparable harm to the structures of the eye if it is accidentally discharged near or into the eye.

The device 200 can include a rapid flash unit (not shown). The rapid flash unit may be, for example, one of the rapid flash units described above with respect to FIGS. 1-5 or some other rapid flash unit. The combination of properly orienting the device, for example, in relation to the propagation axis 900 and propagation envelope 910, and rapidly flashing the device with the rapid flash unit can work together to efficiently deliver energy to the treatment site.

In some embodiments, the light spot at the treatment site has an area of about 1 cm². In general, the light generated by the light source is safe to use near human eyes and will not cause serious or irreparable harm to the structures of the eye if it is accidentally discharged near or into the eye.

The acne treatment device 200 also includes a user input 207 (which in some embodiments is the same as the user input interface 150 described above with respect to FIG. 1) as discussed above with respect to FIGS. 2A-3B. The user input 207 for user control of various operational features. For example, in various embodiments, the user input 207 includes a switch, button (as illustrated), contact, and/or sensor. In some embodiments, the user input 207 causes the device 200 to turn on and/or off, to charge a power supply, to begin or end light flashing, to program the exposure duration, and/or to enter a code to re-activate the device 200.

In the illustrated embodiment, button 207 causes the light source to first charge and then flash. In some embodiments there is a separate power mechanism such as a button or switch to turn on the device 200, while in other embodiments, a single button turns and activates the device 200.

For example, in some embodiments, the user presses the button 207 once in order to power on the device 200, which causes the flashing circuitry to charge (e.g., to charge a storage element, such as a capacitor, of the rapid flash unit). While the circuitry is charging, a status indicator 208 indicates that the device 200 is not ready to be activated. For example, during charging, the status indicator can illuminate to a red color. Once the storage element is charged, the status indicator 208 changes color to indicate that the device 200 is ready for use. For example, the status indicator changes to a green color. In another embodiment, the user presses the button 207 once causing power to be delivered to the device but where the charging circuitry does not begin to charge the storage element of the rapid flash unit. In this embodiment, a subsequent pressing and holding of the button for a specified period of time causes the charging of the storage element. In one embodiment, the device includes circuitry capable of detecting that the amount of charge remaining on the power source (e.g., a battery) is low and/or fully charged. In certain embodiments, another status indicator such as separate LED or set of LEDs indicates the charge status of the power source. In another embodiment, the same status indicator that is used to indicate whether the device is ready to be activated is also used to indicate the charge status of the power source.

Once charged and ready, pressing and releasing the button 207 causes the light source to flash and emit light. When the light source flashes, the flashing circuitry discharges through the light source, and the status indicator 208 again indicates that the device is not ready to be activated. The process can be repeated to deliver additional optical energy to a treatment site, or the device 200 may then be turned off For example, the device 200 may be turned off by pressing and holding the button 207 for a specified duration.

In other embodiments, the user holds the button 207 to fully charge the flashing circuitry, e.g., until the status indicator 208 changes color from red to green. If the user releases the button prior to fully charging the flashing circuitry, the flashing circuitry will discharge, and light will not be generated from the light source. But if the flashing circuitry becomes fully charged, pressing (or pressing and releasing) the button 207 again causes the light source to activate.

In some embodiments, the device 200 includes an illumination light source (not shown) in addition to the therapeutic light source discussed above. The illumination light source may be located in or around the output interface 218 or on another portion of device 200. The illumination light source illuminates portions of the patient's skin in order to identify blemishes and problem areas for therapeutic treatment. The illumination source, for example, may include one or more LEDs, such as white light emitting LEDs. In some embodiments, the device 200 also includes an aiming or pointing mechanism (not shown), such as an aiming beam, or a laser pointer. The aiming mechanism allows the user to more accurately identify problem areas and position and orient the device 200 prior to treatment.

In some embodiments the device 200 includes an aiming beam, an illumination source, or both. The aiming beam or illumination source may be activated by a separate button. For example, in some embodiments a button may be located on the side 222, 224 of the body portion 214 or head portion 212, such that it is accessible by the user's thumb. The user may activate the aiming beam or illumination source by depressing the button on the side with the thumb. Then the user may flash the device 200 using the button 207. In other embodiments, a partial depression of button 207 activates the aiming beam or illumination source and a full depression causes the device 200 to charge the flashing circuitry and activate the therapeutic light source.

Various aspects of the device 200 design provide intuitive use, and help the user orient the output interface 218 prior to activating the light source. This can be important for users that do not have access to a mirror during device 200 usage. For example, the arrangement of the button 207 with respect to the output interface 218 allows for beam propagation axis alignment with respect to a treatment area.

Referring again to FIGS. 9A and 9B, the button 207 may be pressed such that it moves along a button depression axis 920. The button depression axis 920 is substantially aligned with the beam propagation axis 900. This configuration advantageously allows a user to align the beam propagation axis 900 with a treatment site by simply pointing at a desired treatment site or area with the finger used to press the button 207. Once aligned, the user may press the button 207 and to activate the light source, and to cause light from the light source to be directed to the treatment area.

The proper alignment of the device with respect to the treatment area, achieved by the configuration of FIGS. 9A and 9B, along with the rapid flash unit, work in combination to deliver a significant level of energy at the treatment site. For instance, if the device is not properly aligned, too much energy can directed away from the target area. Similarly, even if the device is properly aligned, but does not flash rapidly enough during a treatment cycle to maintain a certain level of energy, too much energy may be lost in between flashes.

Moreover, the general shape of device 200 allows for intuitive and ergonomic application of treatment. For example, the head portion's angulation 940, defined by the device's longitudinal axis 930 and beam propagation axis 900, allows for natural alignment of the user's wrist and fingers when applying treatment to most areas of the body.

As discussed above, the device 200 includes an output interface 218 that serves as the interface between the device 200 and the treatment site, e.g., the user's skin. The output interface 218 can include a transmission surface, mirror, and/or window. In one embodiment, the output interface 218 is disposable. In other embodiments, portions of the output interface 218 are disposable.

In the illustrated embodiment of FIGS. 2A, 2C, 9A, and 9B, the output interface 218 includes a surface 203 which is substantially orthogonal to the beam propagation axis 910. The surface 203 may contact the patient's skin and may be made of metal or plastic material. In some embodiments, the surface 203 is made of a soft rubber. In some embodiments, the surface 203 may be smooth so as to glide across the patient's skin.

The output interface 218 also includes an emission channel 202 through which the therapeutic light is emitted. As shown the emission channel 202 may have an oval shape cross-sectional shape. In other embodiments, the emission channel 202 may have a circular or rectangular cross-sectional shape. As shown, the emission channel 202 forms a recessed treatment chamber having an opening on one side through which the energy (e.g., light and/or heat energy) is emitted. When the device is placed in contact with the skin such that the surface 203 and opening are generally flush with the skin, the treatment chamber becomes closed on all sides. The emission channel 202 of certain embodiments can improve the efficiency and effectiveness of the device. For example, the closed chamber can allow energy (e.g., light and/or heat energy) emitted from the device to be collected and focused such that the energy is more efficiently delivered to and absorbed by the treatment area. As such, the emission channel 202 can lead to more effective treatment and shorter treatment times, among providing other benefits. In certain embodiments, the improved efficiency allows the device to deliver therapeutically significant treatment while using less overall energy, which can lead to other benefits such as longer battery life.

In addition, the output interface 218 can include a heat source, and may optionally include a heat sensor and cooling device, as well. For example, the output interface 218 may include any of the heat sources, sensors, and cooling devices described herein such as those described with respect to FIGS. 19-23C, and may be configured to perform the methods described herein with respect to FIGS. 24A-B.

In the illustrated embodiment, the therapeutic light generated by the light source travels through a transmission window 206 prior to exiting from the device 100. In some embodiments, the transmission window 206 is recessed from an output rim 204 as defined by emission channel 202 and surface 203. The transmission window 206 may be made of various optically-transparent materials including glass, quartz, fluorite or plastic, such as acrylic. The transmission window 206 may include an optical lens which refracts the emitted light to focus it onto a treatment area. Alternatively, the transmission window 206 may have a planar surface. In other embodiments, the transmission window 206 includes an optical coating to filter out undesirable wavelengths from broadband light generated by the light source.

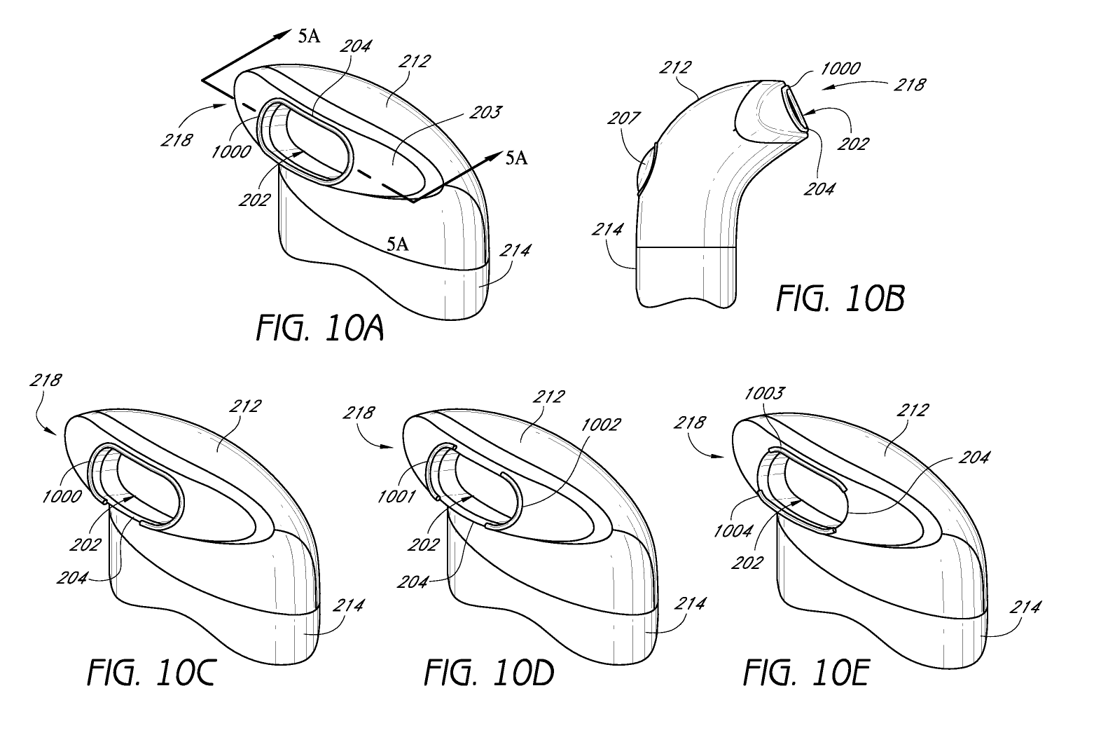

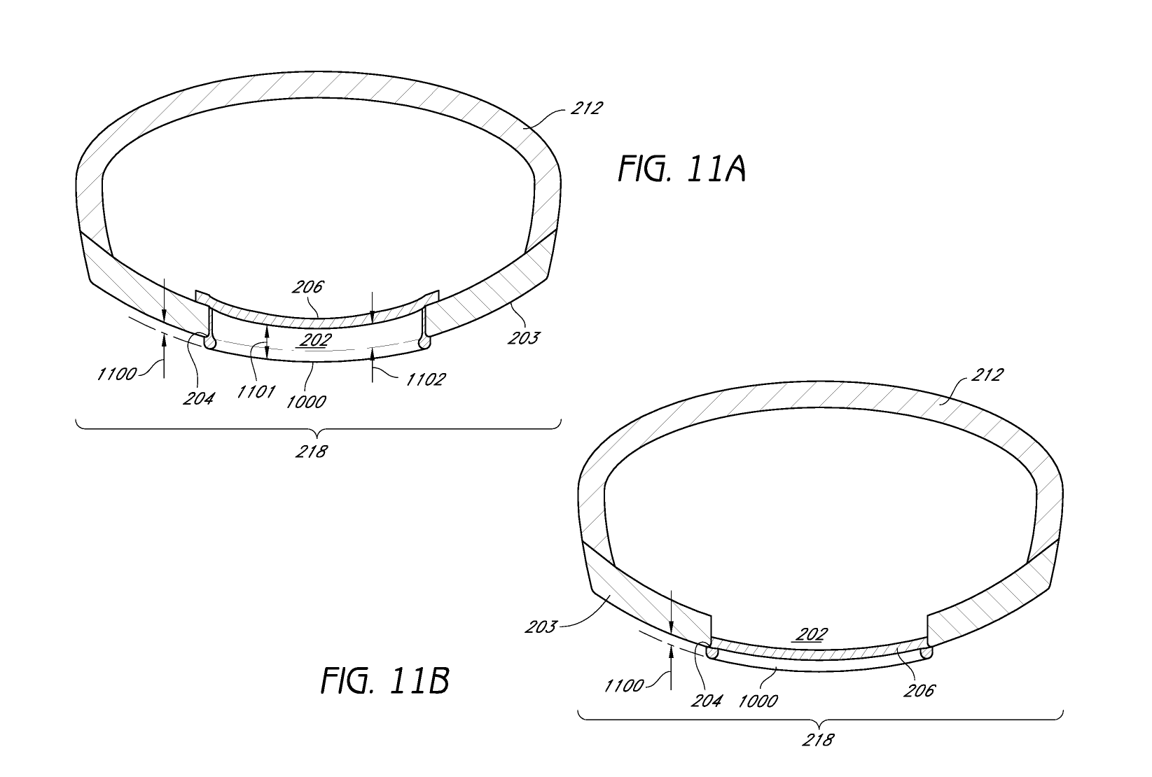

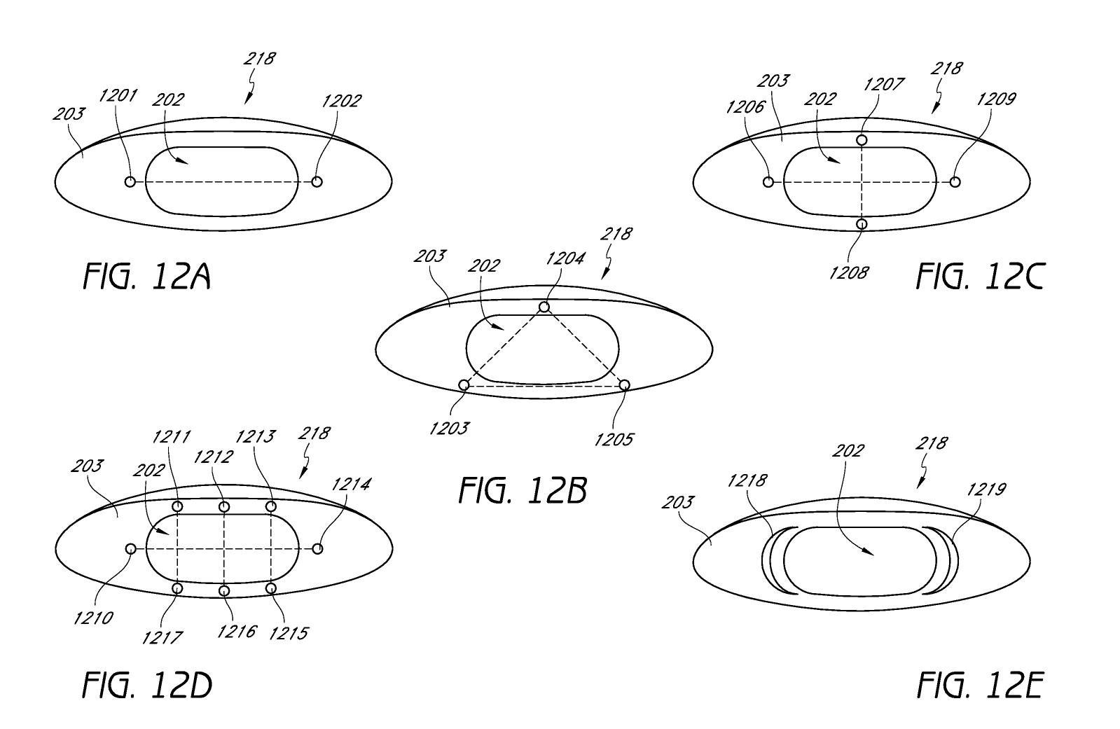

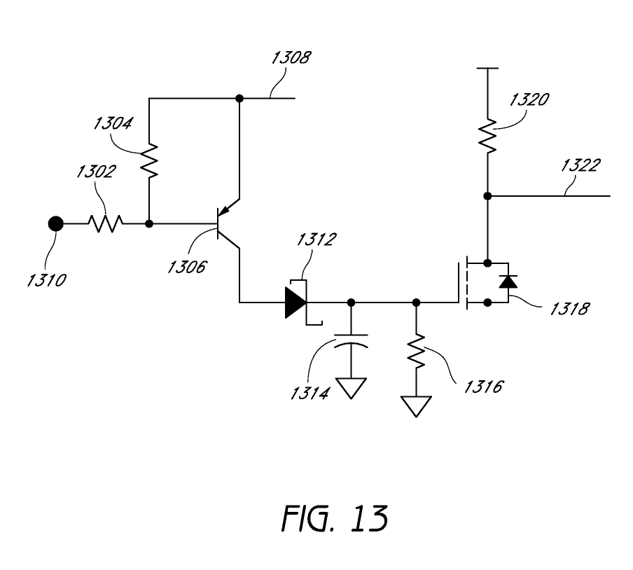

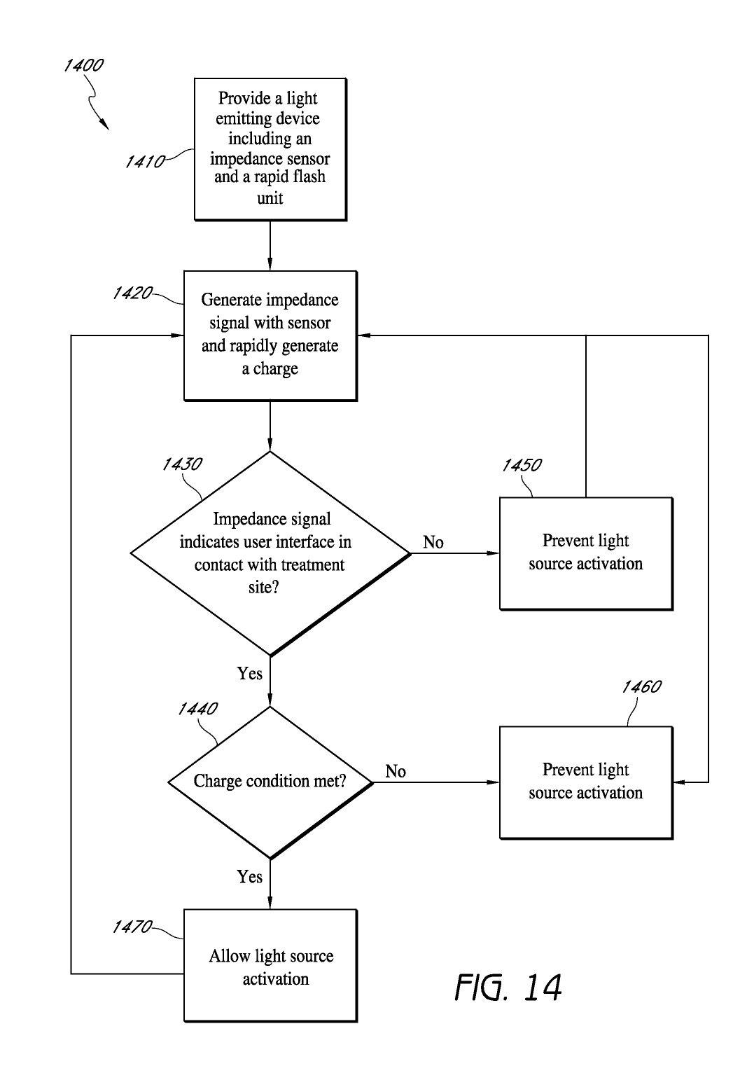

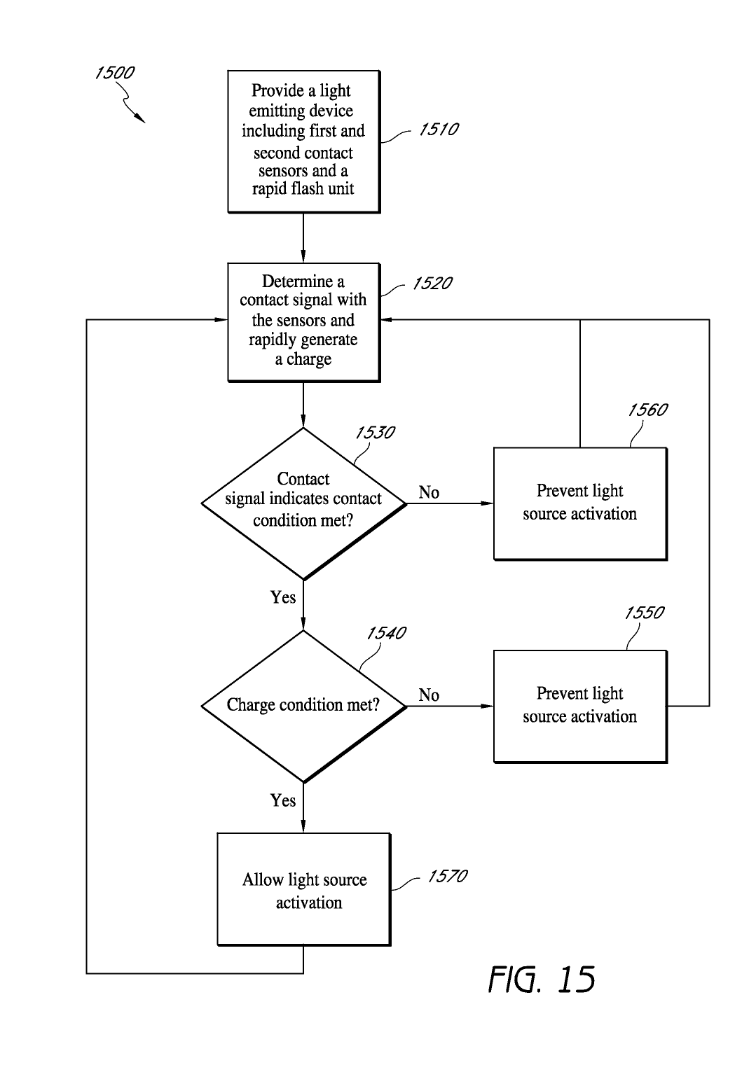

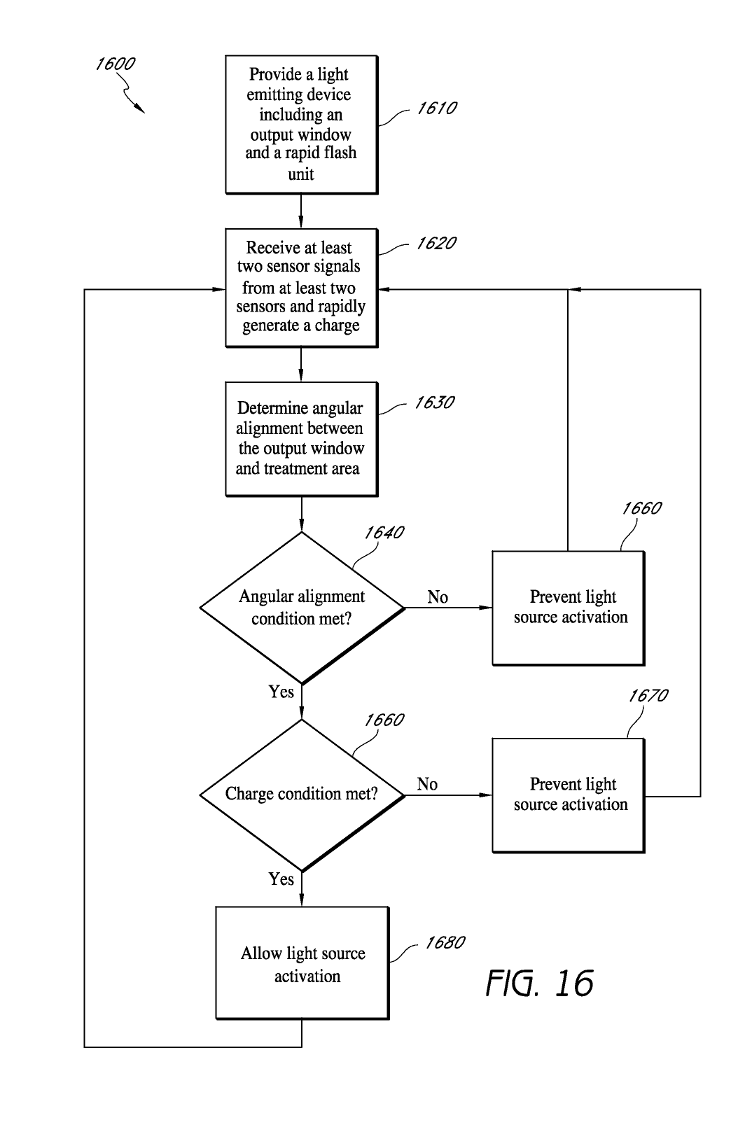

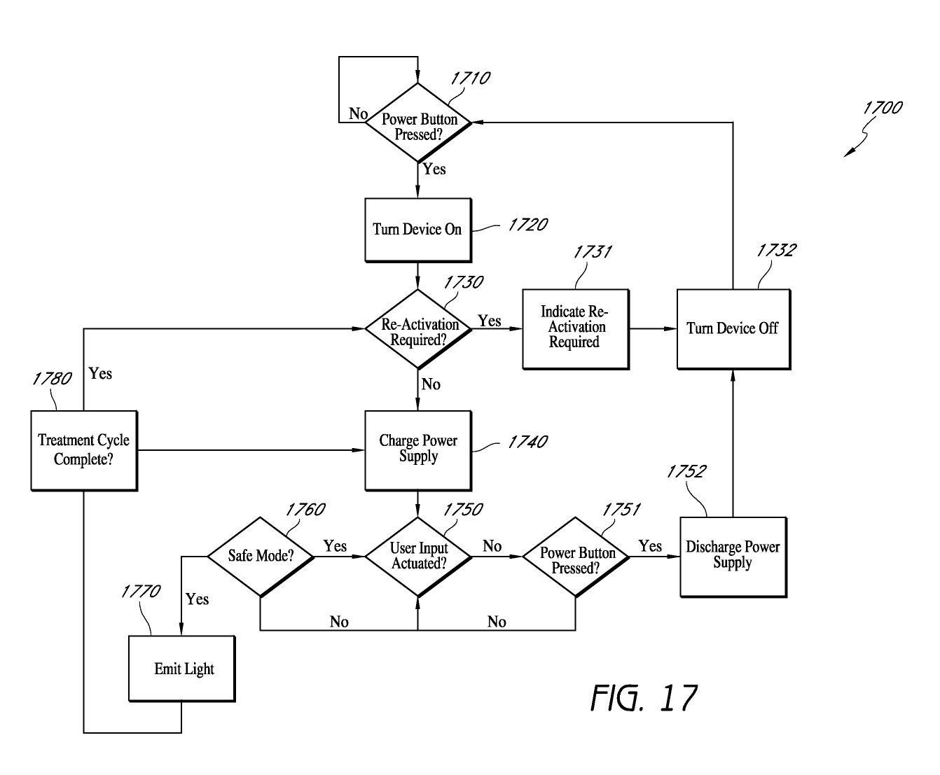

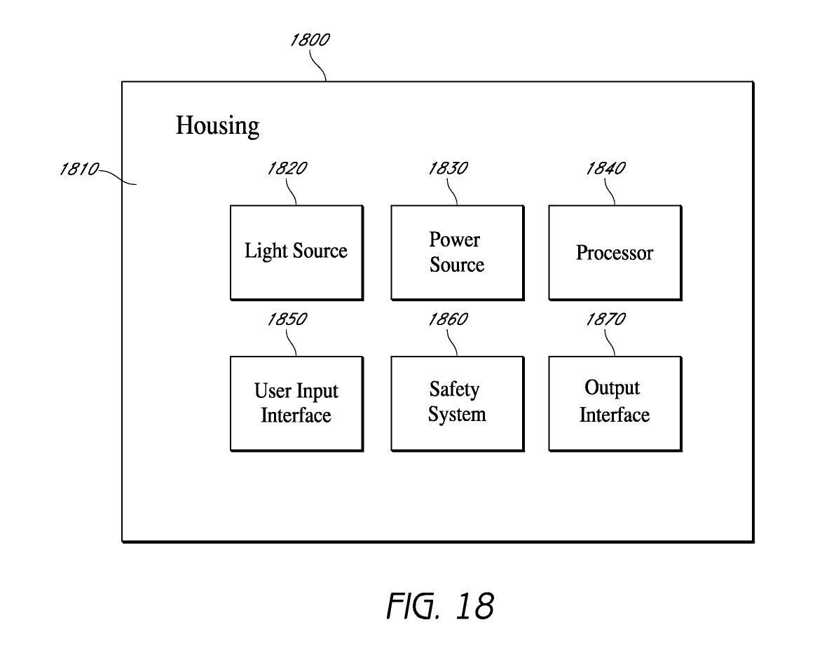

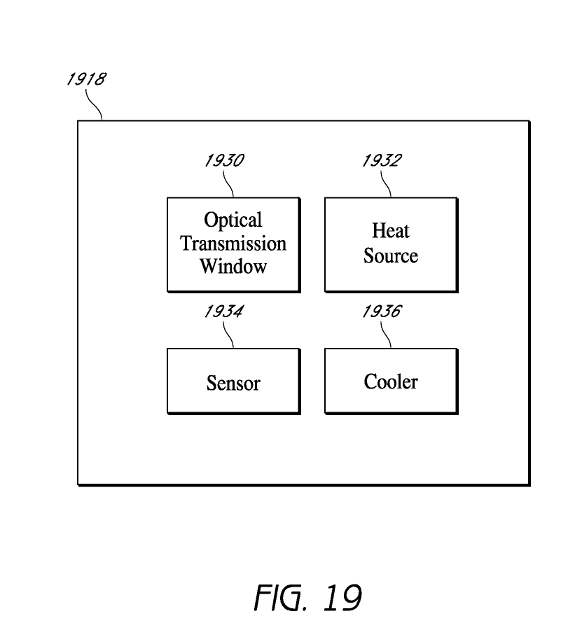

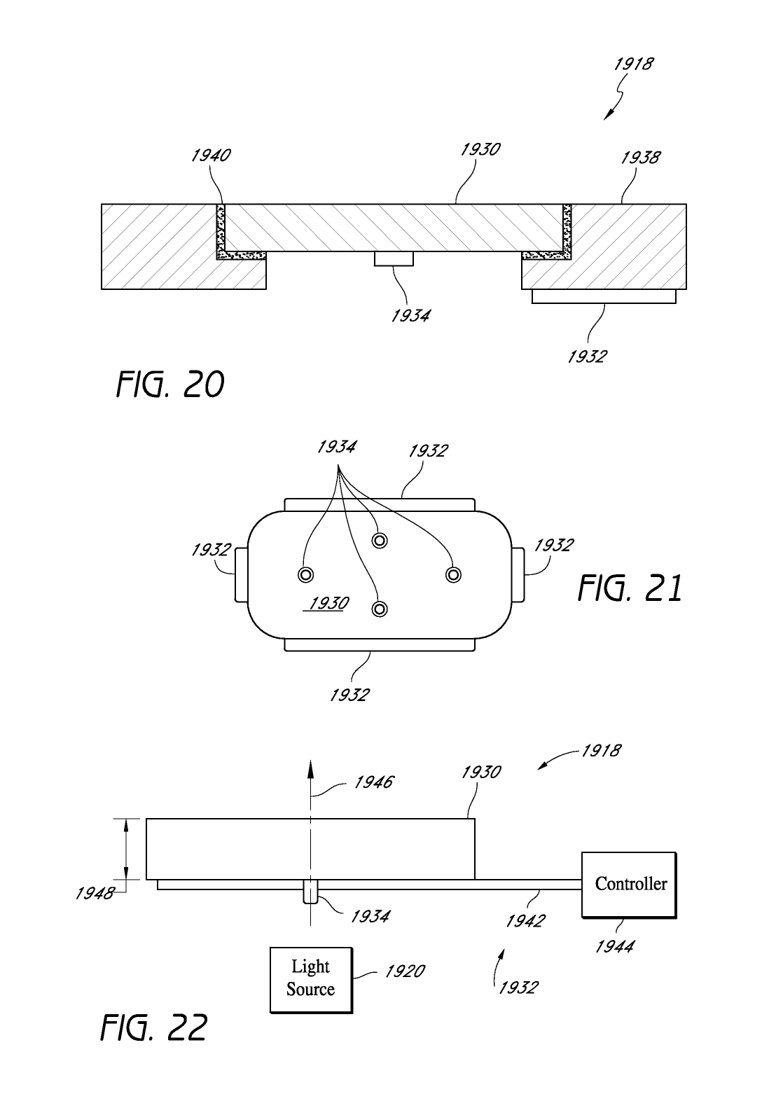

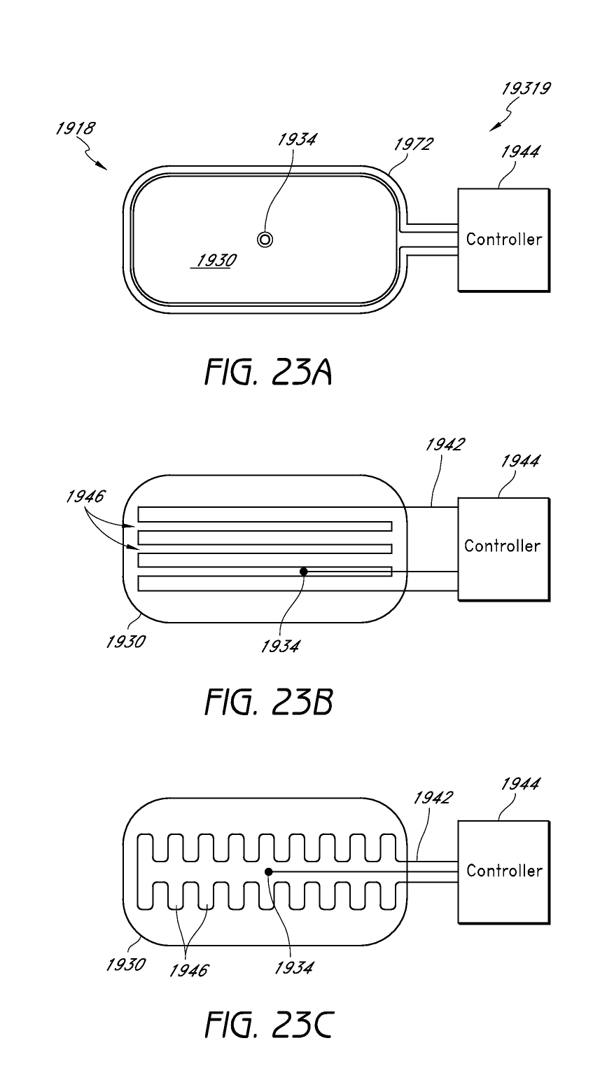

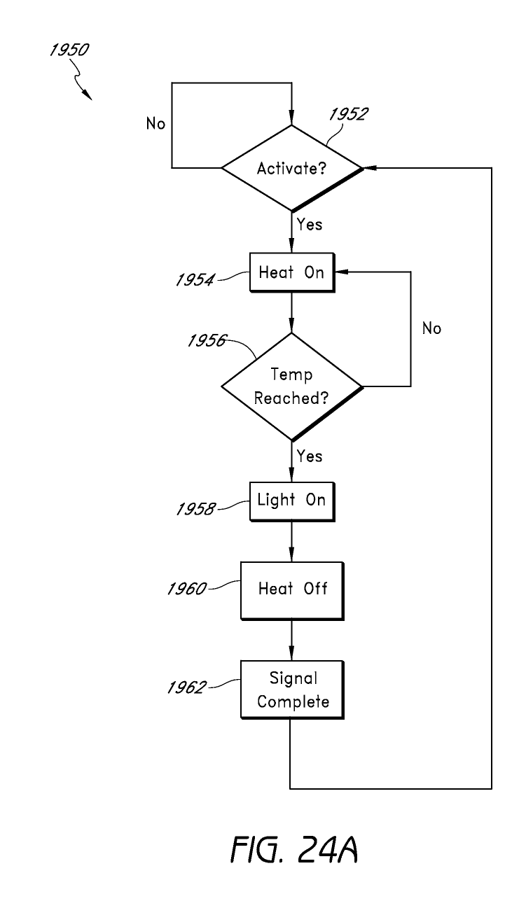

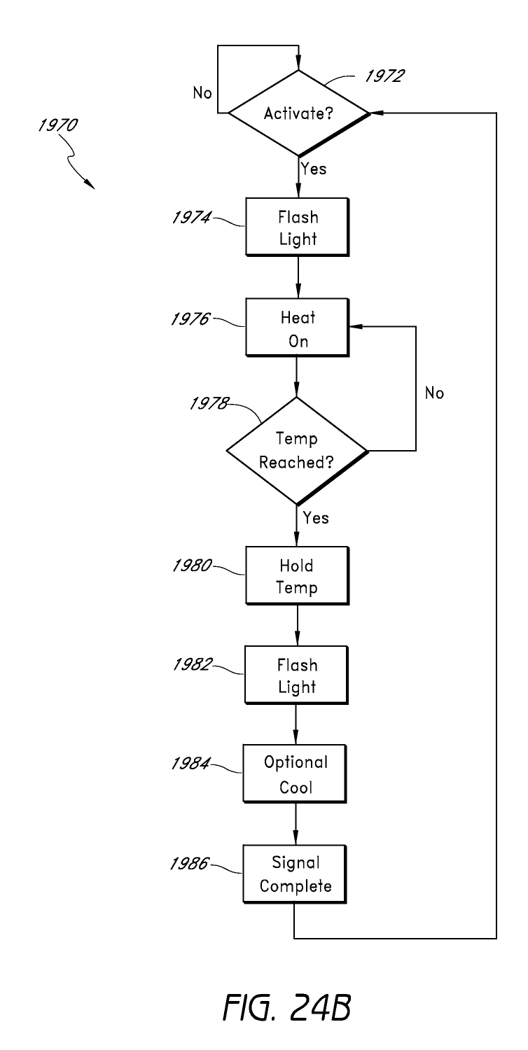

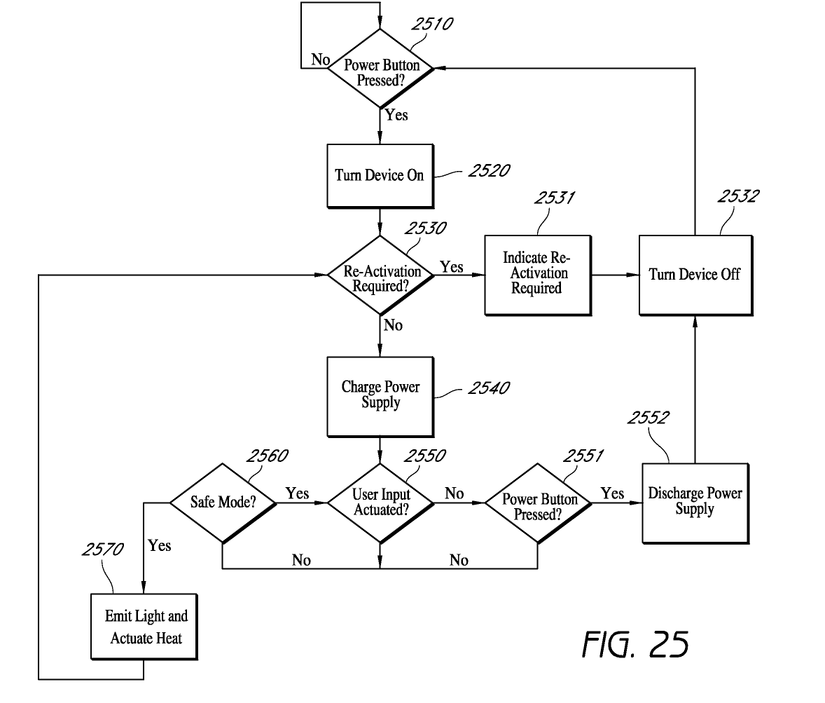

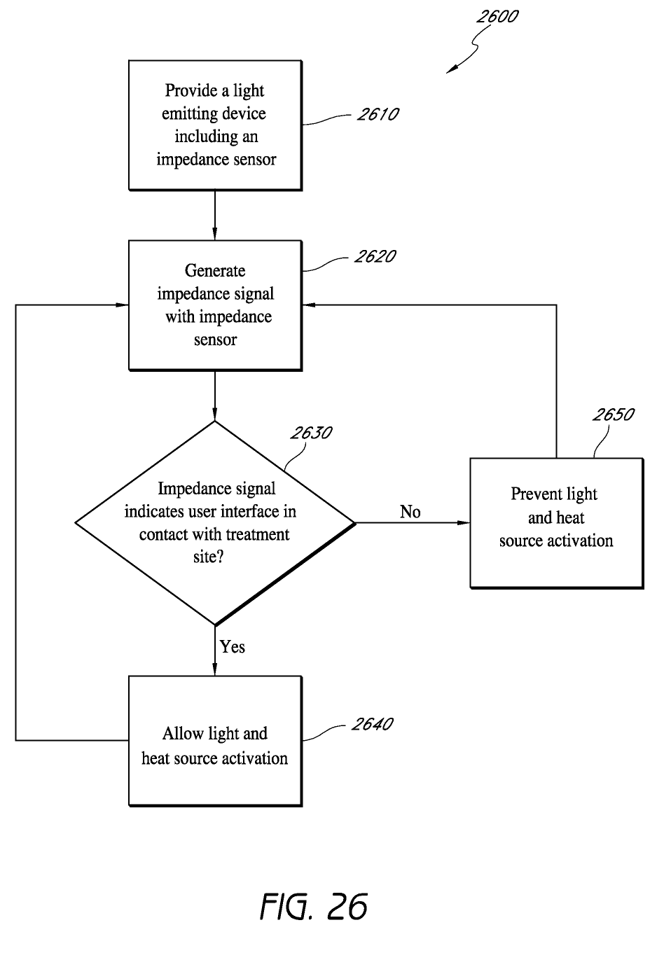

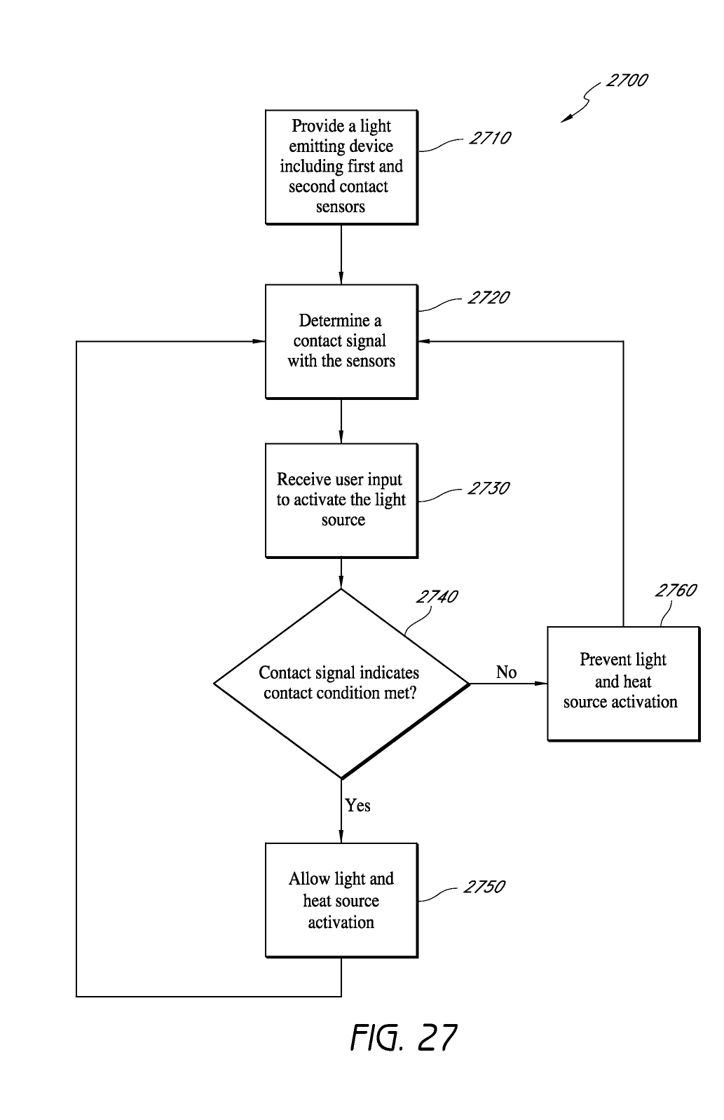

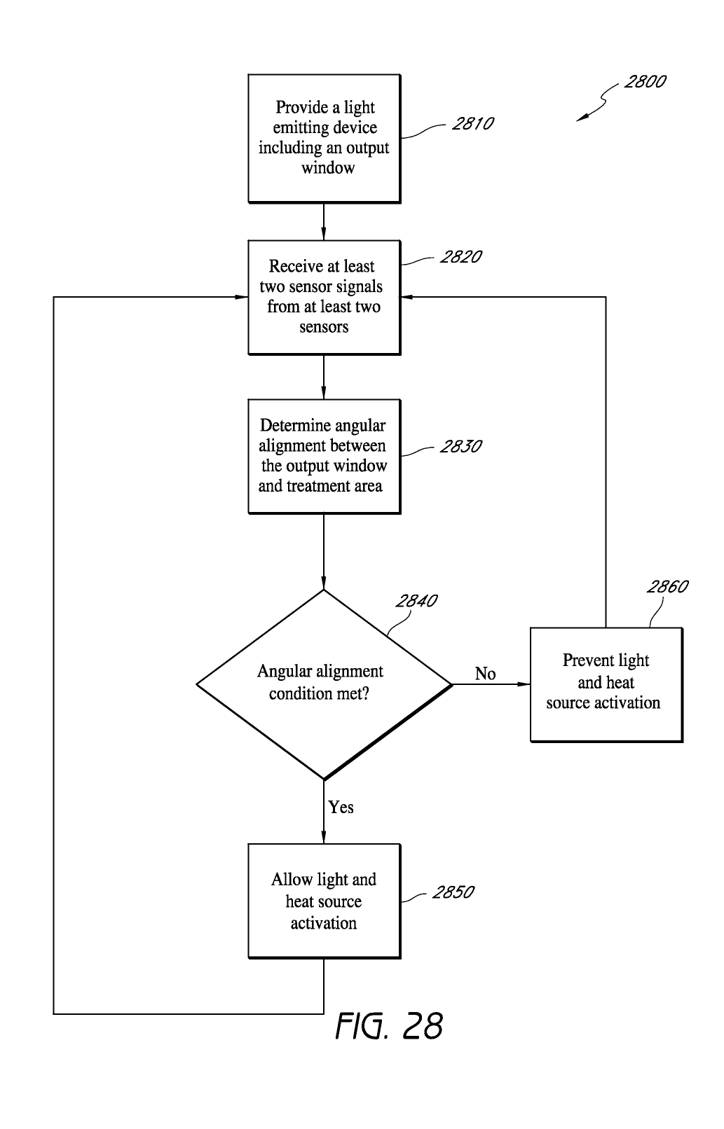

Referring now to FIGS. 10A-E, in some embodiments, the output interface 218 includes a locating ridge 1000 that extends from the surface 203 in the general direction of the beam propagation. As illustrated by FIG. 10A, the locating ridge 1000 may be shaped to conform to the shape of the rim 204 defined by the emission channel 202. In one embodiment, the locating ridge 1000 is made of a soft material, such as rubber, nylon, polyethylene, and/or expanded polytetrafluoroethylene (ePTFE). Certain materials, such as ePTFE, have low friction, lubricious qualities, that provide enhanced comfort to a user when placing the output interface 218 in contact with, and when moved against, the user's skin. The locating ridge 1000 can be made from a soft material that conforms to the patient's features and/or blemishes as the device 200 moves across the skin. In other embodiments, the locating ridge 1000 is made of a plastic or metal. In one embodiment, the locating ridge 1000 is made of an opaque material. The locating ridge 1000 may therefore serve to increase the level of comfort associated with using the device 200, and also to act as a shield to limit, reduce, or prevent light from reaching the eyes of the user or others.

As shown in FIGS. 10A-C, the locating ridge 1000 may be one contiguous member. In other embodiments, as illustrated by FIGS. 10D and 10E, the locating ridge 1000 may include multiple segments 1001, 1002, 1003, 1004. The embodiments illustrated in FIGS. 10D-E show configurations having two segments 1001, 1002 disposed vertically on the sides of the emission channel 202, and two segments 1003, 1004 disposed horizontally on the top and bottom of the emission channel 202, respectively. However, in other embodiments, there may be more than two segments arranged in different configurations. In one embodiment, locating ridge segments are provided on opposite sides of the emission channel 202.

As illustrated by FIG. 10A, the locating ridge 1000 may extend along the entire rim 204 of the emission channel 202. Alternatively, in other embodiments, such as illustrated in FIGS. 10C-E, the segment or segments may only cover or extend along a portion of the rim 204, leaving an opening. The opening or openings may serve to increase the level of comfort associated with using the device 200 by limiting the contact of the locating ridge 1000 with sensitive blemishes when the device 200 is moved across the user's skin.

Embodiments of the device sown in FIG. 10 also include a rapid flash unit (not shown). The rapid flash unit may be, for example, one of the rapid flash units described above or some other rapid flash unit.

As illustrated by FIG. 11A, the transmission window 206 may be recessed or set back from surface 203 of the user interface 218 by a predetermined distance 1102. In addition, the locating ridge 1000 protrudes from output surface 203 a predetermined protrusion distance 1100. When configured in this manner, the transmission window 206 is set back from the user's skin a total distance 1101, which equals the sum of the predetermined distance 1102 and the predetermined protrusion distance 1100, when the device 200 is used.

In another embodiment, the locating ridge 1000 is not provided, and the total distance 1101 from the transmission window 206 to the user's skin (when the device 200 is used) is simply the predetermined distance 1102. In yet other embodiments, as illustrated in FIG. 11B, the transmission window may be aligned flush with the surface 203. As such, the total distance 1101 from the transmission window 206 to the user's skin (when the device 200 is used) is simply the predetermined protrusion distance 1100. In the configuration illustrated by FIG. 11B, if no locating ridge 1000 is present, transmission window is not set back from the user's skin at all, and makes direct contact to the user's skin (when the device 200 is used).

The arrangement of the output surface 203, the transmission window 206, the output rim 204 and the locating ridge 1000 alone or together provide tactile information to the user when the device 200 is used. Tactile information advantageously helps the user determine the position and orientation of the device 200 prior to and during use.

In one embodiment, the emission channel 202 defines a large enough application area to completely surround the acne. In one embodiment, for example, the application area is 1 cm². Moreover, the user interface 218 may define one or more distances 1100, 1101, 1102 between the surface 203 and transmission window 206 deep enough to envelope most blemishes. As the device 200 is moved across the user's skin the user will feel when blemishes are surrounded by the rim 204 and/or contained in the space defined by locating ridge 1000. As such, the user will know when the device 200 is properly positioned with respect to a particular blemish to deliver a therapeutic treatment.

In some embodiments, the output interface 218 and/or various portions thereof (including the locating ridge 1000) are removably attachable and or disposable. In some embodiments, for example, friction mechanisms may be used to allow for removable attachment. In other embodiments, latching mechanisms such as a latch and pocket type of mechanism may be employed. In other embodiments, an adhesive is used to attach the output interface 218 to the device 200, or to attach the locating ridge 1000 to the output interface 218.

In another embodiment, the acne treatment device 200 includes a safety system, such as the safety system 160 described above with respect to FIG. 1. In one embodiment, the safety system includes circuitry and/or sensor that prevent light source activation until a safety condition is realized. For example, in some embodiments, the safety system includes a switch that is activated prior to enabling activation of the light source.

For example, the safety system can include, but is not limited to, a mechanical pressure switch, a contact switch, and/or an electrical switch such as a galvanic response, capacitive, resistance or impedance switch. Such switches are activated when brought into contact with a user's skin, and can prevent the device 200 from emitting light unless the device 200 is in contact with the user's skin. In addition, the switches can prevent light from leaking or being emitted from the device 200 when activated, e.g., flashed.

Embodiments of the device sown in FIG. 11 also include a rapid flash unit (not shown). The rapid flash unit may be, for example, one of the rapid flash units described above with respect to FIGS. 1-5 or some other rapid flash unit.

In some embodiments, the safety system may be used in conjunction with the rapid flash unit to provide safer, more efficient use of the device. For example, in one embodiment, when the safety switch indicates that the device is not in sufficient contact with the treatment area, the rapid flash unit will not charge the device or will discontinue charging the device. This interaction may provide for greater efficient use of the power source (e.g. battery) by not unnecessarily charging the device when it is not ready for use. This arrangement also ensures safe treatment by allowing for rapid flashing during a treatment cycle only when the device is safely in contact with the skin.

Embodiments of safety switch arrangements are illustrated by FIGS. 12A-E. In the various embodiments, safety switch contacts 1201-1219 (sometimes referred to as sensors, or contact sensors) are arranged around the emission channel 202. In some embodiments, the contact arrangement is referred to as a contact array. For example, in the embodiment illustrated in FIG. 12A, two contacts 1201, 1202 are positioned arranged on opposite side of the emission channel 202. Until the patient's skin comes in contact the switch contacts 1201, 1202, the switch is open and the safety system 160 will not allow the device 100 to flash. Once the skin comes in contact with the contacts 1201, 1202 the switch will close and the user may activate the device 200 light source. The device also includes a rapid flash unit. The rapid flash unit may be, for example, one of the rapid flash units described above or some other rapid flash unit.

FIGS. 12B-D illustrate contact configurations employing three, four and eight contacts, respectively. The dotted lines represent linear paths between various combinations of contacts, which when brought into contact with the user's skin, will close the safety switch and enable device 200 activation. For example, in FIG. 12C, the switch will close when either of two contact pairs 1206, 1209 or 1207, 1208 are brought into contact with the user's skin. In one embodiment, the linear paths at least partially traverse the emission channel 202 defined by the user interface 218. By assuring that such contacts are touching the user's skin prior to activation, the device 200 can determine whether the entire planar surface traversing the emission channel 202 is in contact with the user's skin, or if it is inclined at an angle with respect to the user's skin.

The dotted lines serve as possible combinations only and are not meant to limit the number of combinations possible in other embodiments. For example, in other embodiments, simultaneous contact with contacts 1206 and 1207 may also enable device 200 activation.

The embodiments of FIGS. 12A-D include contacts 1201-1214 having circular cross-sectional areas. However, in various embodiments, the safety switch contacts 1201-1214 may be shaped differently. For example, FIG. 12E illustrates a safety switch including crescent shaped contacts 1218, 1219 which are shaped to generally conform to the shape of window 202. The contact shape can be selected to optimize user skin contact for a particular application.

As described above, in some embodiments, the safety switch includes various types of switches including analog- and digital-type electrical switches. The skin is an electrical conductor and therefore has a corresponding resistance. The skin also has a corresponding capacitance. Therefore, the safety system can not only determine contacts are touching skin, but by analyzing the resistance measurements (or signals) obtained from various contacts, the safety system can determine the angle at which the user interface 218 is aligned or tilted with respect to a treatment area on the user's skin.

In other embodiments, the safety system uses the contact sensors or sensor arrays as digital switches. For example, in one embodiment, the safety system monitors the resistance between two contacts, and enables activation of the light source only when the resistance between the appropriate safety switch contacts falls beneath a certain threshold.

For example, with respect to FIG. 12A, when at least one of the contacts 1201, 1202 is not in contact with the skin, the resistance between the contacts 1201, 1202 is very large and the switch is open. In this situation, the device 200 will remain disabled by the safety system. However, when the contacts 1201, 1202 are both brought in contact with the skin, the resistance between them drops to an amount corresponding to resistance of the skin, and the switch will close. The safety system will cause activation, e.g. flashing, of the device 100 to be enabled. Moreover, in certain embodiments, the switch is configured to close when brought into contact with the skin but not when brought into contact with other conductive surfaces having different electrical characteristics.

The safety system includes capacitive sensors and/or switches in some embodiments which detect a change in capacitance indicating that the device is in contact with the skin. For example, referring again to FIG. 12A, the contacts 601, 602 may include one or more capacitors having a corresponding capacitance (e.g., a relatively low capacitance). When one of the contacts 601, 602 are brought into contact with the skin, the capacitance of the skin changes (e.g., increases) the effective capacitance of the respective contact. The safety system detects this change in capacitance on one or more of the contacts 601, 602 and will cause activation, e.g. flashing, of the device 100 to be enabled. In certain embodiments, the safety system only causes activation of the device 100 to be enabled when more than one of the contacts (e.g., both contacts 601 and 602) are in contact with the skin. An advantage of capacitive sensors is that they operate effectively in the presence of facial hair.

One advantage of using electrical sensors such as impedance or capacitive sensors as contact sensors is that the safety system cannot be fooled into thinking that it is in contact with skin by merely pressing down on a mechanical switch. This functionality improves device 200 safety, as it prevents the device 200 from being activated when not contacting skin, which could lead to light emission into a user's or other party's eyes. Product safety is further enhanced by providing two or more sensors, as discussed above.

In other embodiments, the safety switch includes mechanical switches, such as a mechanical pressure switch that closes when a certain amount of pressure is detected by the switch. For example, with respect to FIG. 12A, if a certain threshold pressure is placed on the contacts 1201, 1202, the switch closes, and the safety system 160 causes activation, of the device 200.

The illustrated embodiments include multiple contacts 1201-1219, at least two of which are in contact with the skin to enable activation of the device 200. However, in other embodiments, there may be only one contact or only one contact may need to be in contact with the skin to enable activation of the device. For example, in certain embodiments, a single contact may be placed along rim 204 or locating ridge 1000 or portions thereof Moreover, while the illustrated embodiments show the contact or contacts 1201-1219 disposed on the surface 203, in other embodiments, the contact or contacts 1201-1219 may be located in various other portions of output interface 218. For example, the contact or contacts 1201-1219 may be located underneath the surface 203, on the rim 204, or on or within locating ridge 1000.

In various embodiments, the acne treatment device 200 is configured to limit operability to a predetermined event. For example, the device 200 is generally configured such that is not usable after a certain amount of time, light exposure, light pulses, activations, etc. After the predetermined event occurs, the device 200 can be re-activated. For example, in some cases, the device 200 is re-activated by entering a validation code, or by replacing a part, such as the light source or power supply. In other embodiments, the device 200 is re-activated by downloading an activation code or activation signal from a remote location, such as over the Internet.

In one embodiment, the controller is configured to limit operability of the optical device 200. For example, the controller can be configured to prevent the optical device 200 from emitting light after a predetermined event. In one embodiment, the controller prevents optical device 200 operation after a predetermined number of light flashes, or light emissions are produced (e.g., 50, 100, 250, 500, or 1000 light flashes). In other embodiments, the controller prevents optical device 200 operation after a predetermined time of total light emission (e.g., 5, 10, 15, 30, 60, or 120 minutes). In other embodiments, the controller prevents optical device 200 operation after a predetermined event, such as a predetermined number of device-to-skin contacts (e.g., 50, 100, 250, 500, or 1000 contacts).

In general, the lifetime of the light source can be predetermined and/or set to expire after a preset number of flashes, by mechanical and/or electronic operations, including software and firmware. Such software and/or firmware can be included in the device 200, controller, and/or housing 210. In one embodiment, after reaching the preset maximum number of flashes, the device 200 is re-activated by removing the light source and replacing it with a new light source. In another embodiment, the light source lifetime can be set to expire based on a pre-set number of treatment cycles.

In other embodiments, the device 200 includes an audible signal that warns the user that the system is ready to flash. If within a preset period of time the unit is not placed on to the skin so that the pressure/contact switch is activated the unit discharges, and the flash charge is lost.

In other embodiments, the device 200 includes a timer, such as a timing circuit. The timer is configured to prevent rapid flashing by the user. For example, the timer can provide a delay of about 1, 5, 10, 30, or more than 30 seconds between flashes or light emissions from the light source. The timing circuit can be provided as discreet circuitry and/or implemented with the controller.

In other embodiments, the device 200 includes an indicator, such as an LED, display, icons, microphone, and/or other indicator. In one embodiment, the device 200 emits an audible signal that warns the user that the power is too low on the unit to use.

In other embodiments the device 200 includes at least one security device capable of determining whether the device 200 remains a compliant device. For example, the security device may be configured to assist in the determination of whether the rapid flash unit, light source or output interface 218 are compliant with device 200. The security device may be, for example, a 20K EEPROM well known to those of skill in the art and capable of performing various diagnostic and control functions.

FIG. 13 shows a schematic diagram of a capacitive contact sensor circuit 1300 according to one embodiment of the disclosure. As discussed with respect to FIG. 12, the capacitive contact sensor detects whether the skin is in contact with the sensor. The example sensor circuit 1300 includes an oscillation input 1308, a contact terminal 1310 and an output 1322. The circuit 1300 also includes various resistors 1302, 1304, 1316, 1320, transistor 1306, transistor 1318, capacitor 1314 and diode 1312. An oscillation signal is provided to the input 1308. For example, in one embodiment, a 32 KHz oscillation signal is provided to input 1308. In other embodiments, different frequencies may be used. The contact terminal 1310, for example, may electrically coupled to a contact on the exterior of the device (e.g., one of the contacts of FIG. 12) which can be brought into contact with the skin of a user.

The portion of the circuit including the oscillation input 1308, the resistor 1304, the resistor 1302, the contact terminal 1310 and the exterior contact has an associated, relatively low, capacitance. As such, this portion of the circuit can function as a low pass filter. When the user's skin is not in contact with the skin, the relatively high frequency oscillation input signal is largely blocked by the low pass filter. As a result, the amount of current traveling through the resistor 1304 is relatively small and the corresponding voltage drop across the resistor 1304 is thus also relatively small. Accordingly, the threshold voltage across the transistor 1306 is not met and the transistor 1306 remains switched “off”.

However, once the user's skin contacts the sensor, the capacitance of the skin increases the capacitance of the low-pass filter portion of the circuit, changing the time constant of the filter. The amount of current traveling through the resistor 1304 and the corresponding voltage drop across the resistors 1304 increases such that the threshold voltage across the transistor 1306 is met and the transistor 1306 is switched “on”. The oscillation signal is thus provided to the input of the diode 1312 which performs a half-wave rectification function. The diode 1312 provides a rectified output signal which switches the transistor 1318 “on”. When the transistor 1318 is switched “on”, an increase in the voltage drop across the resistor 1320 and a corresponding change in a signal on the output 1322 indicates to the device that the sensor is contacting the skin. As discussed above with respect to FIG. 12, the device may allow activation in certain embodiments when one of the contacts (e.g., one of the contacts 601 or 602) are in contact with the skin and in other embodiments the device may only allow activation when more than one of the contacts (e.g., both of the contacts 601 and 602) are in contact with the skin.

As shown with respect to FIG. 12, more than one contact sensor circuit 1300 may be present. For example, there may be one contact sensor circuit 1300 corresponding to each contact on the output interface 218. Although described with respect to the example embodiment of FIG. 13, other configurations of a contact sensor circuit 1300 are possible. For example, the frequency of the oscillation signal is different in certain embodiments. In addition, in various configurations, one or more of the individual components shown in FIG. 13 are not included or additional components may be included.

FIG. 14 shows a method 1400 of treating acne with an acne treatment device according to an embodiment of the disclosure. The method 1400 begins at step 1410, in which a light emitting device is provided. In one embodiment, the light emitting device may be the device 100 described above with respect to FIG. 1. In other embodiments, the light emitting device is the device 200 described above with respect to FIG. 2, the light emitting device 1800 described with respect to FIG. 18, or some other light emitting device. The light emitting device includes a light source configured to generate optical energy having a wavelength in a range of from about 400 nm to about 1100 nm. In some embodiments, the light source is the light source 120, described above with respect to FIG. 1, or another light source. The device also includes a rapid flash unit. The rapid flash unit may be, for example, one of the rapid flash units described above with respect to FIGS. 1-5 or some other rapid flash unit.

The device provided by method 1400 further includes a user interface configured to provide a transmission pathway for the optical energy from the light source to a treatment area generally along a beam propagation axis. The transmission pathway may be, for example, transmission path 202, described above with respect to FIG. 2. For example, in some embodiments, the user interface may be the user interface 150 described above with respect to FIG. 1, the user interface 218 described above with respect to FIG. 2, or some other user interface. The user interface includes an electrical impedance sensor, such as, for example, any of the electrical impedance sensors described above, or some other impedance sensor. In other embodiments, the impedance sensor may be another impedance sensor. The device also includes a controller, for example, processor 140 described above with respect to FIG. 1, which is in electrical communication with the light source and electrical impedance sensor.

The method 1400 includes generating an impedance signal with the electrical impedance sensor at step 1420. The device also begins to generate a charge at step 1420 using the rapid flash unit. At decision step 1430, the method 1400 determines whether the user interface is in contact with the treatment site. If at step 1430 the method 1400 determines that the user interface is not in contact with the treatment site, the method 1400 prevents activation of the light source at step 1450 and then the method 1400 then returns to step 1420. On the other hand, if the user interface is in contact with the treatment site, the method 1400 determines if the rapid flash unit has generated a sufficient charge at step 1440. If the method 1400 determines that the charge condition is not met at step 1440, the method prevents light source activation at step 1460 and continues to generate a charge, returning to step 1420. If, on the other hand, the method 1400 determines that the charge condition is met at step 1440, the method 1400 allows activation of the light source at step 1470. For instance, the method 1400 will active the light source when it receives a user input indicating that a user is attempting to activate the light source. For example, the user input may be the user pressing button 207 or some other button or user input mechanism.

In certain embodiments, the steps described above may be performed concurrently or in a different order than shown. For instance, the determination of whether the user interface is in proper contact with the treatment site at step 1430 and whether the charge condition is met at step 1440 may occur generally concurrently or in a different order than shown. In addition, the method 1400 may not begin to generate a charge at all until the method 1400 determines that there is proper contact at step 1430.

The method 1400 may include additional steps not shown in FIG. 14. For instance, the method 1400 may complete a full treatment cycle before it receives the next user input indicating that a user is attempting to activate the light source. For instance, the method 1400 may include activating the light source, generating the optical energy and directing the optical energy to a treatment area. The method 1400 may further include filtering the optical energy prior to delivery to the treatment area. The filtering step may include removing energy having a wavelength outside of a range of from about 400 nm to about 700 nm or by removing energy having a wavelength below about 400 nm. In other embodiments, the method 1400 also includes generating additional optical energy with a second light source. The additional optical energy may include mostly infrared energy. Method 1400 may further include illuminating the treatment area with an illumination light source, such as, for example, any of the illumination light sources described above.

FIG. 15 shows a method 1500 of treating acne with an acne treatment device according to an embodiment of the disclosure. The method 1500 begins at step 1510, in which a light emitting device is provided. The light emitting device may be the device 100 described above with respect to FIG. 1, the light emitting device 200 described above with respect to FIG. 2, the light emitting device 1800 described with respect to FIG. 18, or some other light emitting device. The light emitting device includes a light source configured to generate optical energy having a wavelength in a range of from about 400 nm to about 1100 nm. In some embodiments, the light source is the light source 120, described above with respect to FIG. 1, or another light source.

The acne treatment device further includes a user interface configured to provide a transmission pathway of the optical energy from the light source to a treatment area generally along a beam propagation axis. For example, in some embodiments, the user interface may be the user interface 150 described above with respect to FIG. 1, the user interface 218 described above with respect to FIG. 2, or some other user interface. The user interface is configured to provide a transmission pathway of the optical energy from a light source to a treatment area and includes at least a first and a second contact sensor spaced apart from each other. The transmission pathway may be, for example, transmission path 202, described above with respect to FIG. 2. A linear path from the first contact sensor to the second contact sensor at least partially traverses the transmission pathway. For example, in some embodiments, the contact sensors are the contact sensors described above with respect to FIGS. 6A-6E. For example, with respect to FIG. 6A, the first and second contact sensors may be contacts 601, 602. Moreover, in one embodiment, the linear path is the linear path represented by the dashed line of FIG. 6A. The device also includes a rapid flash unit. The rapid flash unit may be, for example, one of the rapid flash units described above with respect to FIGS. 1-5 or some other rapid flash unit.

The method 1500 further includes determining a contact signal with the contact sensor information provided by the first and second contact sensors at step 1520. The device also begins to generate a charge at step 1520 using the rapid flash unit. At decision step 1530, the method 1500 determines whether a contact condition is met. For example, the method 1500 uses the contact signal to determine whether the first and second contact sensors defining the linear path are in contact with the treatment area. If the contact condition is not met, the method 1500 prevents activation of the light source at step 1560. The method 1500 then returns to step 1520. If, on the other hand, the method 1500 determines that the contact condition is met, the method 1500 determines if the rapid flash unit has generated a sufficient charge at step 1540. If the method 1500 determines that the charge condition is not met at step 1540, the method prevents light source activation at step 1550 and continues to generate a charge, returning to step 1520. If, on the other hand, the method 1500 determines that the charge condition is met at step 1540, the method 1500 allows activation of the light source at step 1570. For instance, the method 1500 will activate the light source when it receives a user input indicating that a user is attempting to activate the light source. For example, the user input may be the user pressing button 207 or some other button or user input mechanism.

In certain embodiments, the steps described above may be performed concurrently or in a different order than shown. For instance, the determination of whether the user interface is in proper contact with the treatment site at step 1530 and whether the charge condition is met at step 1540 may occur generally concurrently or in a different order than shown. In addition, the method 1500 may not begin to generate a charge at all until the method 1500 determines that there is proper contact at step 1530.

The method 1500 may include additional steps not shown in FIG. 15. For instance, the method 1500 may complete a treatment cycle before it receives the next user input indicating that a user is attempting to activate the light source. For instance, the method 1500 may include activating the light source when the contact signal indicates that contact condition is met. The method 1500 may further include receiving a user input to determine if a button has been pressed or released. The method 1500 may also include filtering optical energy after activating the light source. The filtering step may include removing energy having a wavelength outside of a range of from about 400 nm to about 700 nm, or removing energy having a wavelength below about 400 nm. In other embodiments, the method 1500 also includes generating additional optical energy with a second light source. The additional optical energy may include mostly infrared energy. The method 1500 may further include illuminating the treatment area with an illumination light source prior to activating the light source.

FIG. 16 shows a method 1600 of treating acne with an acne treatment device according to another embodiment of the disclosure. The method 1600 begins at step 1610, in which a light emitting device is provided. The light emitting device includes an output window. The output window may be any of the output windows described above, or another output window. The light emitting device may be the device 100 described above with respect to FIG. 1, the light emitting device 200 described above with respect to FIG. 2, the light emitting device 1800 described with respect to FIG. 18, or some other light emitting device. The light emitting device includes a light source configured to generate optical energy having a wavelength in a range of from about 400 nm to about 1100 nm. In some embodiments, the light source is the light source 120, described above with respect to FIG. 1, or another light source.

The device further includes a user interface configured to provide a transmission pathway of said optical energy from said light source to a treatment area generally along a beam propagation axis. For example, in some embodiments, the user interface may be the user interface 150 described above with respect to FIG. 1, the user interface 218 described above with respect to FIG. 2, the user interface 1850 described below with respect to FIG. 19, or some other user interface. The user interface includes at least two sensors. The device also includes a rapid flash unit. The rapid flash unit may be, for example, one of the rapid flash units described above with respect to FIGS. 1-5 or some other rapid flash unit.

At step 1620 the method 1600 receives at least two sensor signals from the sensors. The sensors may be any of the sensors described above or other sensors. The device also begins to generate a charge at step 1620 using the rapid flash unit. Based on the sensor signals, the method 1600 determines the angular alignment between the output window and a treatment area at step 1630. At decision step 1640, the method 1600 determines whether an angular alignment condition is met. For example, the method 1600 determines whether the device is substantially parallel to the surface of the treatment area. In another embodiment, the method 1600 may determine if the output window and treatment area are substantially in contact or if the angle between them is less than a predetermined value.

If the angular alignment condition is not met, the method 1600 prevents activation of the light source at step 1660 and then the method 1600 then returns to step 1620. On the other hand, if the method 1600 determines that the angular alignment condition is met, the method 1600 determines if the rapid flash unit has generated a sufficient charge at step 1650. If the method 1600 determines that the charge condition is not met at step 1650, the method prevents light source activation at step 1670 and continues to generate a charge, returning to step 1620. If, on the other hand, the method 1600 determines that the charge condition is met at step 1650, the method 1600 allows activation of the light source at step 1670. For instance, the method 1600 will active the light source when it receives a user input indicating that a user is attempting to activate the light source. For example, the user input may be the user pressing button 207 or some other button or user input mechanism.

In certain embodiments, the steps described above may be performed concurrently or in a different order than shown. For instance, the determination of whether the user interface is in proper contact with the treatment site at step 1630 and whether the charge condition is met at step 1640 may occur generally concurrently or in a different order than shown. In addition, the method 1600 may not begin to generate a charge at all until the method 1600 determines that there is proper contact at step 1630.

The method 1600 may include additional steps not shown in FIG. 16. For instance, the method 1600 may complete a full treatment cycle before it receives the next user input indicating that a user is attempting to activate the light source. For instance, the method 1600 may include activating the light source, generating the optical energy and directing the optical energy to a treatment area. The method 1600 may further include filtering the optical energy prior to delivery to the treatment area. The filtering step may include removing energy having a wavelength outside of a range of from about 400 nm to about 700 nm or by removing energy having a wavelength below about 400 nm. In other embodiments, the method 1600 also includes generating additional optical energy with a second light source. The additional optical energy may include mostly infrared energy. Method 1600 may further include illuminating the treatment area with an illumination light source, such as, for example, any of the illumination light sources described above.

For instance, the method may complete a treatment cycle before it receives the next user input indicating that a user is attempting to activate the light source.

FIG. 17 shows one embodiment of using an acne treatment device. The light emitting device may be the device 100 described above with respect to FIG. 1, the light emitting device 200 described above with respect to FIG. 2, the light emitting device 1800 described with respect to FIG. 18, or some other acne treatment device. A power button is pressed to turn on the device at step 1710. The device is then turned on at step 1720. The method 1700 then determines if a re-activation of the device is required at step 1730. For example, the device determines if a predetermined number of light flashes have already been provided by the device. If so, the device indicates that re-activation is required at step 1731, and then stops by turning the device off at 1732. Use of the device and light source is prevented.

However, if not, the method charges the power supply at step 1740. For example, the method charges a capacitor. When charged, the method indicates that the device is ready to be used. The method waits until a user input interface is actuated at step 1750. For example, the method waits until the user presses button 207. Prior to actuation, the method monitors the power button to determine if it is pressed again at step 1751. If so, the device discharges the power supply at step 1752 and turns the device off at step 1732.

Otherwise, when the user input interface is actuated, the method checks to see if the safety system is in safe mode at step 1760. For example, the method checks to see if the appropriate contacts of the safety switch are in contact with the user's skin. If not, the method waits until the unit is in safe mode and the user input interface is actuated again at step 1750. If so, the method causes the device to emit a therapeutic light dosage to the user's skin through the output interface at step 1770. After the light is emitted, the method determines if the treatment cycle is completed at step 1780. If the treatment cycle is not completed, the method returns to step 1740 to begin the process of generating a charge for subsequent flashes of the device. If the treatment cycle is completed, however, the method returns to step 1730 to determine whether re-activation of the device is required.

A light emitting therapeutic device 1800 in accordance with one embodiment of the present disclosure is illustrated in FIG. 18. The device 1800 may be the device 100 described above with respect FIG. 1, the device 200 described above with respect to FIG. 2, or some other device.

In the illustrated embodiment, the device 1800 includes a housing 1810 that contains the device's internal components. The mechanical and electronic parts used to operate the device 1800 are contained within the housing 1810. In the illustrated embodiment, a light source 1820, power source 1830, processor 1840 (sometimes referred to as a controller 1840), user input interface 1850, safety system 1860, and output interface 1870 are carried by or contained within the housing 1810 of device the 1800. The device 1800 may also include a heat source (not shown) and may optionally include a sensor for determining a temperature and/or a cooling device. Additional details regarding heat sources, sensors, and cooling devices are provided below. In certain embodiments, the charging of the device 1800 is controlled by a rapid flash unit (not shown) such as one of the rapid flash units described above with respect to FIGS. 1, 3-8 and 13-17. In various embodiments, the power source 130 may be located in the rapid flash unit or in some other part of the device.

As described above with respect to the device 1800 of FIG. 1, light generated by the light source 120 and emitted from the device 1800 may have a therapeutic effect capable of treating Acne and any of a variety of dermatological or skin conditions.

As discussed herein, embodiments of the device 1800 include a first energy source (e.g., the light source 1820) and a second energy source (e.g., the heat source). The device 1800 may also include one or more other energy sources in certain embodiments. In addition, the energy sources may provide therapeutically significant amounts of various forms of energy including optical, electrical, thermal, or some other form of energy. In addition, one or more of the energy sources may deliver more than one form of energy to the treatment area. For example, as discussed, the light source 1820 may deliver optical energy having a photo-dynamic effect and/or thermal energy to the treatment area.

For example, it has been shown that the various acne lesions, such as pimples, whiteheads, acne, etc., may be effectively therapeutically treated by applying an appropriate amount of heat for an appropriate duration. In some cases, acne may be effectively treated by raising the temperature of a treatment area to about 46° C. for about 5-30 seconds. In other cases, acne may be effectively treated by raising the temperature of a treatment area to a temperature in the range of about 40° C. to about 65° C. for a treatment duration in the range of about 5 seconds to about 4 minutes. In all of the embodiments described below, the acne treatment device or method described is configured to provide a therapeutically effective heat dosage to a treatment area for any of the above described treatment durations. In some embodiments, the device is configured to raise the temperature of an output window surface to the treatment temperature. For example, in the embodiments described below, the device may be configured to heat an output window or other portion of the user interface to a treatment temperature, for example, a temperature in the range of about 40° C. to about 65° C., to about 46° C., to about 49° C., to about 55° C. or to about 60° C., for about 5-30 seconds, or in some cases, up to 4 minutes.

FIG. 19 illustrates one embodiment of an output interface 1918 compatible with any of the light-emitting therapeutic device described herein. The output interface 1918 can be the output interface 1870 of FIG. 18, the output interface 170 of FIG. 1, the output interface 218 of FIG. 2, or the output interface of any of the device embodiments described herein. The output interface 1918 includes an optical transmission window 1930 (sometimes referred to as a window 1930), a heat source 1932, a sensor 1934, and a cooler 1936. In other embodiments, the output interface 1918 includes only an optical transmission window 1930 and a heat source 1932. In further embodiments, the output interface 1918 includes a heat source 1932, a sensor 1934, and one or both of a sensor 1932 and a cooler 1936.

Although the embodiments of FIGS. 19-23B describe the output interface 1918 as including a heat source (and optionally a sensor and/or cooling device), it should be understood that in any of these embodiments, these components need not all be completely located within, or part of the output interface. For example, in some embodiments, only the heating element of the heat source is within the output interface 1918. A heat controller (such as a current source to provide current to the heating element) and a cooling device (such as a fan, or heat sink fins) can be located at least partially, if not entirely, outside of the output interface 1918.

The window 1930 can be made from glass or other optically transparent or partially transparent material. In one embodiment, the window 1930 is made from heat absorbing glass. The window 1930 is configured to absorb light in the infrared wavelength. For example, in one embodiment, the window 1930 is made from Schott KG-1, KG-3, or KG-5 glass.

In one embodiment, a window 1930 provides about 90% optical transmission of light having a wavelength between 350 nm and 600 nm and about 0% transmission of light having a wavelength less than about 300 nm or greater than about 850 nm. The window 1930 is generally between one and six mm thick. Window 1930 thickness may be selected to control heating and cooling rates of the window 1930. A thinner window 1930 will generally heat and cool quicker than a thicker window 1930.

A heat source 1932 can include a heater, such as a resistive heater or a film heater, and infrared lamp, or other heat source. The heat source 1932 generally includes a heat element and a heat control unit, has described below. The sensor 1934 is configured to measure the temperature of the window 1930. In one embodiment, the sensor 1934 is a thermistor. The cooler 1936 can include an active cooling device, such as a fan or thermoelectric cooler, a passive cooling device, such as a heatsink or cooling fins, or a combination thereof

FIG. 20 illustrates one embodiment of a user interface 1918 that includes indirect heating from a heat source 1932, which is also compatible with any of the light-emitting therapeutic devices described herein. The user interface 1918 includes a window 1930, heat source 1932, and a sensor 1934, such as those described above. Heat generated by the heat source 1932 is transferred to the window 1930 through a window support 1938. The window support 1938 can also act as a heatsink to help cool the window 1930. The window support 1938 can be made from heat conductive metal, such as aluminum or copper. A thermally conductive compound 1940 can be placed between the window 1930 and the window support 1938 to facilitate heat transfer, as well.

FIG. 21 illustrates another embodiment of a user interface 1918 that includes indirect heating from a heat source 1932, which is also compatible with any of the light-emitting therapeutic devices described herein. In the illustrated embodiment, the user interface 1918 includes multiple heat sources 1932. The heat sources 1932 are positioned around the perimeter of the window 1930. The heat source is 1932 may be mounted directly to the window 1930 or a window support 1938. Providing multiple heat sources 1932 can permit the device to better control heat dissipation through the window 1930. In some embodiments, the multiple heat sources 1932 provide an even distribution of heat through the window 1930. In other embodiments, the multiple heat sources 1932 can be individually controlled to focus or concentrate heat at a particular region of the window 1930.

In the illustrated embodiment, the user interface 1918 includes four heat sources 1930. One heat source 1930 is positioned along each side of the window 1930. In various embodiments, the user interface 1918 includes one, two, 3, 4, 8, or 16 heat sources 1930.

The user interface 1918 also optionally includes one or more sensors 1934. In the illustrated embodiment, the user interface 1918 includes four sensors 1934. Each sensor 1934 is located near each of the heat sources 1932. In various embodiments, the user interface 1918 includes 1, 2, 3, 4, 8, or 16 sensors 1934. In some cases, the user interface 1918 includes the same number of sensors 1934 as heat sources 1930. In other cases, the user interface 1918 includes a different number of sensors 1934 then heat sources 1930.

FIG. 22 illustrates another embodiment of a user interface 1918, which is also compatible with any of the light-emitting therapeutic devices described herein. The user interface 1918 includes a window 1930, heat source 1932, and sensor 1934. The heat source 1932 includes a heat element 1942 and a heat controller 1944. The heat element 1942 extends from the heat controller 1944 to a surface of the window 1930. Energy delivered from the heat controller 1944 is delivered to the window 1930 and converted into heat with the heat element 1942.

A light source 1820 delivers optical energy along an optical transmission axis 1946 of the output interface 1918. The optical transmission axis 1946 is generally aligned in the direction of the window thickness 1948.

A heat element 1942 includes a resistive element, such as a wire, that emits heat as current from the heat source 1944 travels through it. The heat element 1942 may also include an adhesive film, such as Mylar having an adhesive backing, to facilitate attaching the heat element 1942 to a surface of the window 1930. In one embodiment, the heat element 1942 is at least partially transmissive to optical energy, or light, emitted from the light source 1820. By using and at least partially transmissive heat element 1942, the output interface 1918 can advantageously provide both heat and optical energy to a treatment site.

FIGS. 23A-23C illustrate various embodiments of user interface is 1918 that include a window 1930, heat source 1932, and in some cases sensor 1934, and which are also compatible with any of the light-emitting therapeutic devices described herein. The heat source 1932 generally includes a heating element 1942 and a controller 1944. In FIG. 22A, the heating element 1942 extends around the perimeter of the window 1930. The configuration illustrated in the embodiment of FIG. 22A advantageously provides a uniform thermal gradient from all sides of the window 1930. This can help avoid hot or cold spots from forming in the heated window 1930.

In FIG. 23B, the heating element 1942 extends back and forth across the surface of the window 1930 in a zigzag pattern. Gaps 1946, or open spaces, are defined between segments of the zigzagging heat element 1942. These gaps 1946 advantageously provide the ability of light from a light source to pass through the heat element 1942 while the heat element 1942 provides heat to the window 1930. In FIG. 7C, the heat element 1942 extends across the surface of the window 1930 in a sinusoidal pattern. Gaps 1946 within the sinusoidal pattern advantageously allow light from a light source to pass through the heat element 1942, as well.

Providing gaps 1946 between heating elements 1942, or between portions of a heating element 1942, can advantageously allows more efficient window 1930 heating and quicker device operation. For example, extending a heating element 1942 across a window 1930, such as in a zig-zag or sinusoidal pattern will generally allow the window 1930 to heat uniformly more quickly than by merely providing a heating element 1942 off to one side of the window 1930, or even around the window 1930 perimeter.

However, the heating element 1942 can interfere with the transmission of light from a light source to a treatment site by, for example, absorbing, scattering, reflecting, and/or blocking light transmission. Therefore, providing gaps 1946 between heating elements 1942 (or heating element 1942 segments), light can pass through the window unaffected at least in part, to thereby allow both quick window 1930 heating and clinically effective light transmission.

Also, in some embodiments, the heating element 1942 is applied to a window 1930 in a manner designed to block a predetermined amount or percentage of light generated by the light source. Such blocking provides a convenient filtering or regulating mechanism to assure that only the clinically desired light dosage is applied to the treatment site.

FIG. 24A and FIG. 24B illustrate embodiments of methods of providing optical and thermal energy with a light emitting therapeutic device. The method 1950 of FIG. 24A begins at step 1952, where the method 1950 determines if a light emitting therapeutic device has been activated. If not the method 1950 waits for activation. In one embodiment, activation results from a user of a light-emitting therapeutic device pressing a button to turn the device on, or to initiate a treatment. If activation has occurred, the method 1950 continues to step 1954. At step 1954 the method 1950 turns on a heat source, such as any of the heat sources described herein. The method 1950 continues to step 1956, where the method 1950 determines if a desired or predetermined temperature has been reached. For example, in some embodiments, the method 1950 pre-heats the window of the user interface of a light-emitting therapeutic device prior to activating a light source. In some embodiments, the desired temperature is about 46 degrees Celsius. Alternatively, the desired temperature can be any of the treatment temperatures described herein.

If the desired or predetermined temperature has not been reached, the method 1950 returns to step 1954. However, if the desired or predetermined temperature has been reached, the method 1950 continues to step 1958. At step 1958, the method 1950 activates a light source (e.g., a flashlamp, LED, or any other therapeutic light source described herein) while heat continues to be delivered from the heat source. In addition, at step 1958, the method 1950 may continue to deliver heat from the heat source for a predetermined therapeutic period, such as any of the therapeutic treatment periods described herein (e.g., 5 seconds, 10 seconds, 20 seconds, etc.). The method 1950 then continues to step 1960, where the heat source is turned off At step 1960, the method 1950 may optionally activate a cooling device, such as a thermoelectric cooler or a fan.

The method 1950 then continues to step 1962, where the method 1950 indicates that the treatment is complete. For example, in one embodiment, the method 1950 provides a signal to the user at step 1962. The signal may be activating a light (such as an LED indicator, etc.), emitting a noise (such as a beep, tone, chime, etc.), and/or activating a buzzer or other vibrating device. In other embodiments, the method 1950 does not provide a signal and skips step 1962. The method 1950 then returns to step 1952 to determine if the light-emitting therapeutic device has been activated.

The method 1970 of FIG. 24B begins at step 1972, where the method 1970 determines if a light-emitting therapeutic device has been activated. If not, the method 1970 waits for activation. If activation has occurred, the method 1970 continues to step 1974. At step 1974 the method 1970 activates a light source, such as any of the light sources described herein. For example, in one embodiment, the method 1970 flashes a flashlamp at step 1974. The method 1970 continues to step 1976, where the method 1970 then activates a heat source. At step 1978, the method 1970 determines if a desired or predetermined temperature has been reached.

If the desired or predetermined temperature has not been reached, the method 1970 returns to step 1976. If the desired or predetermined temperature has been reached, the method 1970 continues to step 1980. At step 1980, the method 1970 holds the temperature at the desired or predetermined level. For example, the method 1970 can turn the heat source on and off in response to a sensor signal to maintain the temperature of a user interface of a light-emitting therapeutic device at the desired or predetermined level.

The method 1970 may then return to step 1972, or may continue with one or more of the steps illustrated in FIG. 24B. For example, in one embodiment, the method 1970 continues to step 1982, where the method 1970 activates a light source. The light source may be activated more than one time, for example, multiple flashes may be emitted from the light source. The method 1970 may continue to step 1984, where the method 1970 activates a cooling device to cool a light-emitting therapeutic device's user interface. The method 1970 may then continue to step 1986, where the method 1970 provides a signal to the user indicating that treatment is completed. The signal may be activating a light (such as an LED indicator, etc.), emitting a noise (such as a beep, tone, chime, etc.), and/or activating a buzzer or other vibrating device.

FIG. 25 shows one embodiment of using an acne treatment device, such as any of the acne treatment devices describe above. A power button is pressed to turn on the device at step 2510. The device is then turned on at step 2520. The method then determines if a re-activation of the device is required at step 2530. For example, the device determines if a predetermined number of light flashes have already been provided by the device. If so, the device indicates that re-activation is required at step 2531, and then stops by turning the device off at 2532. Use of the device and light source is prevented.

However, if not, the method charges the power supply at step 2540. For example, the method charges a capacitor. When charged, the method indicates that the device is ready to be used. The method waits until a user input interface is actuated at step 2550. For example, the method waits until the user presses button 207. Prior to actuation, the method monitors the power button to determine if it is pressed again at step 2551. If so, the device discharges the power supply and shuts off at step 2550.

Otherwise, when the user input interface is actuated, the method checks to see if the safety system is in safe mode at step 2560. For example, the method checks to see if the appropriate contacts of the safety switch are in contact with the user's skin. If not, the method waits until the unit is in safe mode and the user input interface is actuated again at step 2550. If so, the method causes the device to emit a therapeutic light dosage to the user's skin through the output interface at step 2570. After the light is emitted, the method returns to step 2530 to determine whether re-activation of the device is required.

Alternatively, the method 2500 may also activate the device's heat source at step 2570. For example, the method 2500 can simultaneously activate, or alternate activation between, the light and heat sources. In one embodiment, the method 2500 causes light and heat to be emitted from a device according to the steps described above with respect to FIGS. 24A-B.

FIG. 26 shows a method 2600 of treating acne with an acne treatment device according to another embodiment. The method 2600 begins at step 2610, in which a light emitting device is provided. The light emitting device can be the device 1800 described above with respect to FIG. 18 or the light emitting device 100 described above with respect to FIG. 1. In other embodiments, the light emitting device is the device 200 described above with respect to FIG. 2, or some other light emitting device. The light emitting device includes a light source configured to generate optical energy having a wavelength in a range of from about 400 nm to about 1200 nm, and a heat source configured to deliver heat to a user interface. In some embodiments, the light source is the light source 1820, described above with respect to FIG. 18, or another light source, such as any of the light sources described herein.

The device provided by method 2600 further includes a user interface configured to provide a transmission pathway for the optical energy from the light source to a treatment area generally along a beam propagation axis. The transmission pathway may be, for example, transmission path 202, described above with respect to FIG. 2. For example, in some embodiments, the user interface may be the user interface 150 described above with respect to FIG. 1, the user interface 218 described above with respect to FIG. 2, or some other user interface. The user interface includes an electrical impedance sensor, such as, for example, any of the electrical impedance sensors described above, or some other impedance sensor. In other embodiments, the impedance sensor may be another impedance sensor. The device also includes a controller, for example, processor 140 described above with respect to FIG. 1, which is in electrical communication with the light source and electrical impedance sensor.

The method 2600 includes generating an impedance signal with the electrical impedance sensor at step 2620. At decision step 2630, the method 2600 determines whether the user interface is in contact with the treatment site. If the user interface is in contact with the treatment site, the method 2600 allows activation of the light and heat sources and generation of the optical and heat energy at step 2640. The method 2600 then returns to step 2620. On the other hand, if at step 2630 the method 2600 determines that the user interface is not in contact with the treatment site, the method 2600 prevents activation of the light and heat sources at step 2650. The method 2600 then returns to step 2620.

The method 2600 may include additional steps not shown in FIG. 26. For instance, the method 2600 may include activating the light source, generating the optical energy and directing the optical energy to a treatment area. The method 2600 may further include filtering the optical energy prior to delivery to the treatment area. The filtering step may include removing energy having a wavelength outside of a range of from about 400 nm to about 700 nm or by removing energy having a wavelength below about 400 nm. In other embodiments, the method 2600 also includes generating additional optical energy with a second light source. The additional optical energy may include mostly infrared energy. Method 2600 may further include illuminating the treatment area with an illumination light source, such as, for example, any of the illumination light sources described above.

In addition, the method 2600 may activate the device's heat source at step 2640 in a variety of manners. For example, the method 2600 can simultaneously activate, or alternate activation between, the light and heat sources. In one embodiment, the method 2600 causes light and heat to be emitted from a device according to the steps described above with respect to FIGS. 24A-B.

FIG. 27 shows a method 2700 of treating acne with an acne treatment device according to an embodiment of the disclosure. The method 2700 begins at step 2710, in which a light emitting device is provided. The light emitting device may be the device 1800 described above with respect to FIG. 18, device 100 described above with respect to FIG. 1, the light emitting device 200 described above with respect to FIG. 2, or some other light emitting device. The light emitting device includes a light source configured to generate optical energy having a wavelength in a range of from about 400 nm to about 1200 nm and a heat source. In some embodiments, the light source is the light source 120, described above with respect to FIG. 1, or another light source.

The acne treatment device further includes a user interface configured to provide a transmission pathway of the optical energy from the light source to a treatment area generally along a beam propagation axis. For example, in some embodiments, the user interface may be the user interface 1850 described above with respect to FIG. 18, the user interface 150 described above with respect to FIG. 1, the user interface 218 described above with respect to FIG. 2, or some other user interface. The user interface is configured to provide a transmission pathway of the optical energy from a light source to a treatment area and includes at least a first and contact sensor spaced apart from each other. The transmission pathway may be, for example, transmission path 202, described above with respect to FIG. 2. A linear path from the first contact sensor to the second contact sensor at least partially traverses the transmission pathway. For example, in some embodiments, the contact sensors are the contact sensors described above with respect to FIGS. 12A-6E. For example, with respect to FIG. 12A, the first and second contact sensors may be contacts 601, 602. Moreover, in one embodiment, the linear path is the linear path represented by the dashed line of FIG. 12A.

The method 2700 further includes determining a contact signal with the contact sensor information provided by the first and second contact sensors at step 2720. The method 2700 then receives user input at step 2730 indicating that the user is attempting to activate the light source. For example, the user input may be the user pressing button 207 or some other button or user input mechanism. At decision step 2740 the method 2700 determines whether a contact condition is met. For example, the method 2700 uses the contact signal to determine whether the first and second contact sensors defining the linear path are in contact with the treatment area. If the contact condition is met, the device allows light source and heat source activation at step 2750. The method 2700 then returns to step 2720. On the other hand, at step 2760, if the contact condition is not met, the method 2700 prevents activation of the light source and the heat source. The method 2700 then returns to step 2720.

The method 2700 may activate the device's heat source at step 2750 in a variety of manners. For example, the method 2700 can simultaneously activate, or alternate activation between, the light and heat sources. In one embodiment, the method 2700 causes light and heat to be emitted from a device according to the steps described above with respect to FIGS. 24A-B.

The method 2700 may include additional steps not shown in FIG. 27. For instance, the method 2700 may include activating the light source when the contact signal indicates that contact condition is met. The method 2700 may further include receiving a user input to determine if a button has been pressed or released. The method 2700 may also include filtering optical energy after activating the light source. The filtering step may include removing energy having a wavelength outside of a range of from about 400 nm to about 700 nm, or removing energy having a wavelength below about 400 nm. In other embodiments, the method 2700 also includes generating additional optical energy with a second light source. The additional optical energy may include mostly infrared energy. The method 2700 may further include illuminating the treatment area with an illumination light source prior to activating the light source.

FIG. 28 shows a method 2800 of treating acne with an acne treatment device according to another embodiment of the disclosure. The method 2800 begins at step 2810, in which a light emitting device is provided. The light emitting device includes an output window. The output window may be any of the output windows described above, or another output window. The light emitting device may be the device 1800 described above with respect to FIG. 18, the device 280 described above with respect to FIG. 1, the light emitting device 200 described above with respect to FIG. 2, or some other light emitting device. The light emitting device includes a light source configured to generate optical energy having a wavelength in a range of from about 400 nm to about 1200 nm and a heat source. In some embodiments, the light source is the light source 1820, described above with respect to FIG. 18, the light source 120 described above with respect to FIG. 1, or another light source.

The device further includes a user interface configured to provide a transmission pathway of said optical energy from said light source to a treatment area generally along a beam propagation axis. For example, in some embodiments, the user interface may be the user interface 1850 described above with respect to FIG. 18, the user interface 150 described above with respect to FIG. 1, the user interface 218 described above with respect to FIG. 2, or some other user interface. The user interface includes at least two sensors.

At step 2820 the method 2800 receives at least two sensor signals from the sensors. The sensors may be any of the sensors described above or other sensors. Based on the sensor signals, the method 2800 determines the angular alignment between the output window and a treatment area at step 2830. At decision step 2840, the method 2800 determines whether an angular alignment condition is met. For example, the method 2800 determines whether the device is substantially parallel to the surface of the treatment area. In another embodiment, the method 2800 may determine if the output window and treatment area are substantially in contact or if the angle between them is less than a predetermined value.

If the angular alignment condition is met, the method 2800 allows activation of the light source and heat source at step 2850. The method then returns to step 2820. On the other hand, if the angular alignment condition is not met, the method 2800 prevents light source and heat source activation at step 2860. The method then returns to step 2820. The method 2800 may further include illuminating the treatment area with an illumination light source. The illumination light source may include any of the illumination light sources described above.

The method 2800 may activate the device's heat source at step 2850 in a variety of manners. For example, the method 2800 can simultaneously activate, or alternate activation between, the light and heat sources. In one embodiment, the method 2800 causes light and heat to be emitted from a device according to the steps described above with respect to FIGS. 24A-B.

Although the foregoing invention has been described in terms of certain preferred embodiments, other embodiments will be apparent to those of ordinary skill in the art from the disclosure herein. Moreover, the described embodiments have been presented by way of example only, and are not intended to limit the scope of the inventions. Indeed, the novel methods and systems described herein may be embodied in a variety of other forms without departing from the spirit thereof Accordingly, other combinations, omissions, substitutions and modifications will be apparent to the skilled artisan in view of the disclosure herein.

1. A method of maintaining a therapeutically significant level of energy at a treatment area, the method comprising:
determining current provided to a primary winding of a fly-back transformer, the fly-back transformer comprising said primary winding and a secondary winding;
charging an energy storage device by discharging energy from said secondary winding into said energy storage device when said measured current is within a predetermined range;
repeating said determining and said discharging until stored energy with said energy storage device is within a predetermined discharge range;
triggering a flashlamp when said stored energy is within the predetermined discharge range such that said stored energy is discharged across said flashlamp;
emitting light from said flashlamp into a treatment area as a result of said triggering, said light comprising a plurality of wavelengths in a range of from about 400 nm to about 100 nm; and
repeating said charging and triggering a predetermined number of times during a treatment period to raise and maintain a temperature at said treatment area to a predetermined therapeutic level.

2. The method of claim 1, wherein said energy storage device comprises at least one capacitor.

3. The method of claim 1, wherein said fly-back transformer further comprises a core, and wherein said predetermined range corresponds to flux saturation of said core.

4. The method of claim 1, wherein said predetermined discharge range comprises about 300 Vdc.

5. The method of claim 1, wherein said triggering comprises ionizing gas contained in said flashlamp.

6. The method of claim 1, wherein said light comprises a plurality of wavelengths in a range of from about 400 nm to about 700 nm.

7. The method of claim 1, wherein said predetermined number of times is six times.

8. The method of claim 1, wherein said treatment period is about two seconds.

9. The method of claim 1, wherein said treatment area comprises tissue containing Acne Vulgaris bacteria.

10. The method of claim 1, wherein said predetermined therapeutic level is about 49 degrees Celsius.

11. The method of claim 1, wherein said predetermined therapeutic level is at least 49 degrees Celsius.

12. The method of claim 1, wherein said repeating said charging and triggering is sufficient to raise and maintain an energy density at the treatment area to a predetermined therapeutic energy density.

13. The method of claim 12, wherein the predetermined therapeutic energy density is about 6 J/cm² at the treatment area.

14. The method of claim 13, wherein the predetermined therapeutic energy density is at least 6 J/cm² at the treatment area.

15-28. (canceled)

29. A light-emitting therapeutic device for treating acne, comprising:
a fly-back transformer;
an energy storage device coupled to an output of said fly-back transformer;
a flashlamp coupled to said energy storage device; and
a controller, said controller configured to flash said flashlamp, by delivering energy from said energy storage device to said flashlamp, a sufficient number of times within a predetermined treatment period to raise the temperature of a treatment area having acne to at least 49 degrees Celsius during said treatment period.

30. The device of claim 29, wherein said sufficient number of times is six times.

31. The device of claim 29, wherein said predetermined treatment period is about two seconds.

32. A method of treating acne with a light-emitting therapeutic device, comprising:
charging an energy storage device using a fly-back transformer;
triggering a flashlamp to discharge energy stored with said energy storage device across said flashlamp, wherein said triggering causes said flashlamp to emit therapeutic light; and
repeating said charging and triggering a sufficient number of times within a predetermined treatment period to raise the temperature of a treatment area having acne to at least 49 degrees Celsius during said treatment period with said therapeutic light.

33. The method of claim 32, wherein said sufficient number of times is six times.

34. The device of claim 32, wherein said predetermined treatment period is about two seconds.

35-106. (canceled)
